(19) United States
(12) Patent Application Publication
(10) Pub. No.: US 2006/0250578 A1
(43) Pub. Date: Nov. 9, 2006

(54) Systems and methods for controlling, monitoring, and using remote applications

(76) Inventors: Garrick G. Pohl, Holland, MI (US); Corbin L. Collet, Saugatuck, MI (US); Sylvester J. Bentley, Holland, MI (US)

Correspondence Address: J. SCOTT SOUTHWORTH ATTORNEY AT LAW, P.O. BOX 1287, FRAMINGHAM, MA, 01701, US

(21) Appl. No.: 11/418,310

(22) Filed: May 4, 2006

Related U.S. Application Data
(60) Provisional application No. 60/678,560, filed on May 6, 2005.

(51) Int. Cl. A61B 3/14

(52) U.S. Cl. 351/210

(57) ABSTRACT

A system for controlling, monitoring, and using remote applications. The system includes a mobile communications device in communication with plural gateways to plural environments having plural applications and remote devices. The control software of the mobile communications device provides a graphic user interface (GUI) that presents a multi-environment menu having icons for the plural environments, single environment menus having icons for applications within each single environment, and application submenus having icons for application messages (e.g., commands and/or other options) for each application. The gateway for each environment provides two-way communication between the mobile communications device and the remote devices.

### RELATED APPLICATIONS

This application claims the benefit of U.S. Provisional Patent Application No. 60/678,560, filed on May 6, 2005, the entire teachings of which are incorporated herein by reference. This application is also related to concurrently filed U.S. patent application No. ______, titled “Systems and Methods for Managing Messages for Controlling, Monitoring, and Using Remote Applications,” Attorney Docket No. CI-002, the entire teachings of which are incorporated herein by reference.

### BACKGROUND

Control, monitoring, and application use systems are designed to allow users to remotely interface with functional devices. These systems consist of hard-line or wireless systems topologies. The hard-line systems utilize phone lines or cable infrastructures to carry information from the user to functional devices. The hard-line systems utilize a computer interface to execute commands and monitor status. The wireless systems typically utilize short range RF (radio frequency) or cellular infrastructures for communication. The short-range systems require an RF control device, while the cellular systems typically utilize tone/numeric input, voice input or operator interface to execute commands. Some systems allow for status check, while many are one-way control systems.

### SUMMARY OF THE INVENTION

In one aspect, the invention features a method for controlling, monitoring, and using remote applications over communications connections to a mobile communications device. The method includes providing a multi-environment menu to a display of the mobile communications device. The multi-environment menu presents one or more environment icons. Each environment includes one or more remote applications providing access to one or more remote devices. The method also includes providing a selected environment menu to the display of the mobile communications device based on a selection of one of the presented environment icons. The selected environment menu presents one or more application icons. The method also includes providing a selected application submenu to the display of the mobile communications device based on a selection of one of the presented application icons. The selected application submenu presents one or more remote device icons. The method also includes initiating one or more messages from the mobile communications device to a selected remote device to invoke one or more remote actions based on a selection of one of the presented remote device icons.

In one embodiment, the method also includes providing a selected remote device submenu to the display of the mobile communications device based on the selected remote device icon. The selected remote device submenu presents icons for one or more remote actions. The method also includes initiating one or more messages to the selected remote device based on a selection of one of the presented remote action icons.

In another embodiment, the method also includes providing a response on the display of the mobile communications device received from the selected remote device in response to the one or more messages initiated from the mobile communications device to the selected remote device.

The method, in a further embodiment, includes providing an alert to the display of the mobile communications device in response to one of the remote devices initiating the alert. In another embodiment, the method includes transferring the one or more messages to a gateway associated with a selected remote application indicated by the selected application icon. The method also includes, in one embodiment, transferring the one or more messages to a server for transfer to a selected remote application indicated by the selected application icon.

In another embodiment, the one or more environment icons include one or more of an automotive environment icon, a home environment icon, a business environment icon, a real estate environment icon, a remote water meter environment icon, a remote gas meter environment icon, a medical device environment icon, a machinery environment icon, a rental vehicle environment icon, a fire alarm environment icon, and a farm irrigation environment icon.

In a further embodiment, the one or more application icons include one or more of a door lock application icon, an ignition switch application icon, an electrical outlet application icon, an environmental control application icon, a climate control application icon, a security control application icon, a video camera application icon, a water meter application icon, a gas meter application icon, a medical device application icon, a mechanical device application icon, a rental vehicle application icon, a fire alarm application icon, and a farm irrigation application icon.

In another embodiment, the mobile communications device includes one or more navigation controls and one or more selection controls. The method further includes operating one of the navigation controls to navigate to one of the presented environment icons, and operating one of the selection controls to select that presented environment icon; operating one of the navigation controls to navigate to one of the presented application icons, and operating one of the selection controls to select that presented application icon; and operating one of the navigation controls to navigate to one of the presented remote device icons, and operating one of the selection controls to select that presented remote device icon.

In another aspect, the invention features a server accessing a mobile communications device over communications connections. The server includes control software for controlling, monitoring, and using remote applications; a processor for executing the instructions of the control software; and a communications module. The processor is configured by the instructions of the control software to provide a multi-environment menu to a display of the mobile communications device, to provide a selected environment menu to the display based on a selection of one of the presented environment icons, and to provide a selected application submenu to the display based on a selection of one of the presented application icons. The multi-environment menu presents one or more environment icons. Each environment includes one or more remote applications providing access to one or more remote devices. The selected environment menu presents one or more application icons. The selected application submenu presents one or more remote device icons for invoking one or more remote actions based on a selection of one of the presented remote device icons. The communications module receives one or more messages initiated from the mobile communications device and transfers one of the multi-environment menu, the environment menus, and application submenus to the display of the mobile communications device in response to the one or more received messages. In another embodiment, the server is either an administrative server or a communications server.

In another aspect, the invention features a server accessing a mobile communications device over communications connections. The server includes means for controlling and monitoring remote applications and a communications module. The means for controlling and monitoring remote applications provide a multi-environment menu to a display of the mobile communications device, a selected environment menu to the display based on a selection of one of the presented environment icons, and a selected application submenu to the display based on a selection of one of the presented application icons. The multi-environment menu presents one or more environment icons. Each environment includes one or more remote applications providing access to one or more remote devices. The selected environment menu presents one or more application icons. The selected application submenu presents one or more remote device icons for invoking one or more remote actions based on a selection of one of the presented remote device icons. The communications module receives one or more messages initiated from the mobile communications device and transfers one of the multi-environment menu, the environment menus, and application submenus to the display of the mobile communications device in response to the one or more received messages. In another embodiment, the server is either an administrative server or a communications server.

In one embodiment, the means for controlling and monitoring remote applications provides a selected remote device submenu to the display of the mobile communications device based on the selected remote device icon. The selected remote device submenu presents icons for the one or more remote actions. The communications module receives the one or more messages to the selected remote device based on a selection of one of the presented remote action icons.

In another embodiment, the means for controlling and monitoring remote applications provides a response on the display of the mobile communications device received from the selected remote device in response to the one or more messages initiated from the mobile communications device to the selected remote device. The means for controlling and monitoring remote applications, in a further embodiment, provide an alert to the display of the mobile communications device in response to one of the remote devices initiating the alert. In another embodiment, the communications module transfers the one or more messages to a gateway associated with a selected remote application indicated by the selected application icon.

In another aspect, the invention features a computer program propagated signal product embodied in a propagated medium having instructions for control software for controlling, monitoring, and using remote applications. The instructions, when carried out by a processor of a mobile communications device, configure the mobile communications device to perform the steps of providing a multi-environment menu to a display of the mobile communications device, providing a selected environment menu to the display based on a selection of one of the presented environment icons, providing a selected application submenu to the display based on a selection of one of the presented application icons, and initiating one or more messages from the mobile communications device to a selected remote device to invoke one or more remote actions based on a selection of one of the presented remote device icons. The multi-environment menu presents one or more environment icons. Each environment includes one or more remote applications providing access to one or more remote devices. The selected environment menu presents one or more application icons. The selected application submenu presents one or more remote device icons.

In one embodiment, the computer program propagated signal product includes configuring the mobile communications device to provide a selected remote device submenu to the display of the mobile communications device based on the selected remote device icon, and to initiate one or more messages to the selected remote device based on a selection of one of the presented remote action icons. The selected remote device submenu presents icons for the one or more remote actions.

The computer program propagated signal product, in another embodiment, includes configuring the mobile communications device to provide a response on the display of the mobile communications device received from the selected remote device in response to the one or more messages initiated from the mobile communications device to the selected remote device.

In another embodiment, the computer program propagated signal product configures the mobile communications device to provide an alert to the display of the mobile communications device in response to one of the remote devices initiating the alert.

The computer program propagated signal product, in a further embodiment, configures the mobile communications device to transfer the one or more messages to a gateway associated with a selected remote application indicated by the selected application icon.

In another embodiment, the computer program propagated signal product configures the mobile communications device to transfer the one or more messages to a server for transfer to a selected remote application indicated by the selected application icon.

In another aspect, the invention features a method for controlling, monitoring, and using remote applications over communications connections to a server. The method includes receiving at a server one or more messages from a mobile communications device based on a selection of a remote device icon from a selected application submenu selected from one or more application icons provided by an environment menu selected from one or more environment icons provided by a multi-environment menu, presented on a display of the mobile communications device; transferring one or more received messages to a destination associated with a remote device indicated by the selected remote device icon; receiving at the server a response based on the one or more received messages transferred to the destination; and transferring the received response to the mobile communication device.

In one embodiment, the method further includes providing the received response to the display of the mobile communications device. In another embodiment, the server is either an administrative server or a communications server. The destination, in another embodiment is either a mobile communication device, a gateway, an application, or a remote device. The method, in a further embodiment, includes receiving at the server an alert initiated by the destination and transferring the alert to the mobile communications device.

In another embodiment, the method includes transferring the one or more received messages to a gateway associated with the selected application menu indicated by the selected application icon. The gateway provides access to one or more remote applications having one or more remote devices.

The remote device icon, in another embodiment, is either a door lock icon, an ignition switch icon, an electrical icon, a climate control icon, a video camera icon, a water meter icon, a gas meter icon, a medical device icon, a mechanical device icon, a rental vehicle icon, a fire alarm icon, or a farm irrigation icon.

In another aspect, the invention features a server accessing a mobile communications device over communications connections. The server includes a processor and instructions for managing messages for controlling, monitoring, and using remote applications. The instructions for managing messages configure the processor to receive one or more messages from a mobile communications device based on a selection of a remote device icon from a selected application submenu selected from one or more application icons provided by an environment menu selected from one or more environment icons provided by a multi-environment menu, presented on a display of the mobile communications device; to transfer one or more received messages to a destination associated with a remote device indicated by the selected remote device icon; to receive at the server a response based on the one or more received messages transferred to the destination; and to transfer the received response to the mobile communication device.

In one embodiment, the instructions for managing messages configure the processor to provide the received response to the display of the mobile communications device. In another embodiment, the server is either an administrative server or a communications server. The destination, in a further embodiment, is either a mobile communication device, a gateway, an application, or a remote device.

In another embodiment, the instructions for managing messages configure the processor to receive at the server an alert initiated by the destination and to transfer the alert to the mobile communications device.

### BRIEF DESCRIPTION OF THE DRAWINGS

The above and further advantages of this invention may be better understood by referring to the following description in conjunction with the accompanying drawings, in which like numerals indicate like structural elements and features in various figures. The drawings are not necessarily to scale, emphasis instead being placed upon illustrating the principles of the invention.

FIG. 1 is a schematic drawing that depicts a system for controlling, monitoring, and using remote applications, according to the principles of the invention.

FIG. 2 is a schematic drawing that depicts an environment including a gateway module and applications, according to the principles of the invention.

FIG. 3 is a schematic drawing that depicts an environment including a gateway module, a functional module, and applications, according to the principles of the invention.

FIG. 4 is a schematic drawing that depicts an environment including a gateway module, multiple functional modules, and applications, according to the principles of the invention.

FIG. 5 is a flowchart that depicts a procedure for communicating with a remote device from the display of a mobile communications device, using multi-environment and environment menus, according to the principles of the invention.

FIG. 6 is a flowchart that depicts a procedure for communicating with a remote device from the display of a mobile communications device, using a multi-environment menu, environment menu, and application submenu, according to the principles of the invention.

FIG. 7 depicts a networked system for managing remote applications (e.g., controlling, monitoring and using the applications), according to the principles of the invention.

FIG. 8 is a flowchart that depicts procedure for transferring multi-environment messages through a message server between a mobile communication device and a remote device, according to the principles of the invention.

FIG. 9 is a flowchart that depicts a procedure for transferring multi-environment data between a server and a destination, according to the principles of the invention.

FIG. 10 is an illustration of a GUI (graphical user interface) for multiple environments, according to the principles of the invention.

FIG. 11 is an illustration of the automotive environment menu of FIG. 10, according to the principles of the invention.

FIG. 12 is an illustration of the home environment menu of FIG. 10, according to the principles of the invention.

FIG. 13 is a diagram that depicts an access procedure for gaining access to the multi-environment monitoring and control system for the multiple environments GUI of FIG. 10, according to the principles of the invention.

FIG. 14 is a diagram that depicts a lighting control procedure for accessing a lighting application submenu for the multiple environments GUI of FIG. 10, according to the principles of the invention.

FIG. 15 is a diagram that depicts a car door locks procedure for accessing a car door locks application submenu for the multiple environments GUI of FIG. 10, according to the principles of the invention.

FIG. 16 is a flowchart that depicts an installation procedure for registering and installing the MCMS (multi-environment control and monitoring system) software.

FIG. 17 is an illustration of a customer account home display screen, according to the principles of the invention.

FIG. 18 is an illustration of a change control mapping screen related to the customer account home screen of FIG. 17, according to the principles of the invention.

FIG. 19 is an illustration of an account activity screen related to the customer account home screen of FIG. 17, according to the principles of the invention.

FIG. 20 is an illustration of a software download screen related to the customer account home screen of FIG. 17, according to the principles of the invention.

FIG. 21 is a flowchart that depicts a procedure for business door control in a business facilities environment, according to the principles of the invention.

FIG. 22 is a flowchart that depicts a procedure for real estate door control in a real estate environment, according to the principles of the invention.

FIG. 23 is a flowchart that depicts a procedure for meter control and monitoring in a measurement environment, according to the principles of the invention.

FIG. 24 is a flowchart that depicts a procedure for controlling and monitoring a medical device in a patient environment, according to the principles of the invention.

FIG. 25 is a flowchart that depicts a procedure for controlling and monitoring a mechanical device in a machine environment, according to the principles of the invention.

FIG. 26 is a flowchart that depicts a procedure for vehicle control in a rental environment, according to the principles of the invention.

FIG. 27 is a flowchart that depicts a procedure for a fire alarm notification in a building environment, according to the principles of the invention.

FIG. 28 is a flowchart that depicts a procedure for controlling and monitoring equipment in a farm environment, according to the principles of the invention.

FIG. 29 is an illustration that depicts a multi-environment vehicle GUI, according to the principles of the invention.

### DETAILED DESCRIPTION

A control, monitoring, and application use communication system is typically made up of 1) a transmitter 2) a receiver and 3) a functional module. 1) The transmitter is the device that is used to communicate commands or to monitor a device. These transmitters can receive information in cases where two-way communication is present (transceiver). 2) The receiver is a remote device that receives the incoming commands from the transmitter and determines what function to perform. In the case of wireless data transmission, these devices can contain cellular hardware. 3) The functional modules receive input from the receiver and perform functions. The functional modules control devices, such as door locks, thermostats, car alarms, etc. In cases where two-way communication is present the functional module can be a transceiver.

Conventional communication systems are typically designed to monitor and control one environment, such as a system that controls features of an automobile. A user must become familiar with and use multiple control and Monitoring systems to control different environments (e.g., one system for an automotive environment and another system for the home environment). Conventional control and monitoring communications systems typically do not provide interfaces that are user friendly and/or graphic user interfaces (GUI's). The user must adapt to different types or styles of interfaces for different environments.

A control, monitoring, and application use communication system implemented according to the principles of the invention addresses the above problems and includes many or all of the following features:

1) A mobile communication device (e.g., cellular telephone) is the interface point and is mobile so that the user can control devices remotely.

2) A single control device (e.g., the mobile communication device or cellular telephone) allows users to control and monitor plural applications including consumer and/or commercial devices.

3) The signal range for the control device is increased over some conventional systems by utilizing existing mobile service networks. These devices can be controlled in the same way a person makes a mobile to mobile telephone call today.

4) Graphic user interface (GUI) software allows the user to interface with devices in multiple environments via the mobile communication device. This GUI software allows for two-way communication, allowing the user to control, monitor, and use the remote devices in the multiple environments.

5) A transceiver provides communication to mobile communication devices (e.g., wireless control devices or cellular telephones). This “black box” will act as a switchboard or gateway to direct incoming signals to the appropriate remote devices. Together, the gateway and functional modules comprise a network for the user to monitor/control. Typically, each environment has its own gateway, and the user can access multiple gateways for multiple environments from the multi-environment GUI of the mobile communication control device (e.g., cellular telephone).

6) Functional modules allow for the activation and monitoring of plural applications within consumer and commercial environments. Examples of applications include, but are not limited to: door locks, light switches, ignition switches, environmental controls and security systems

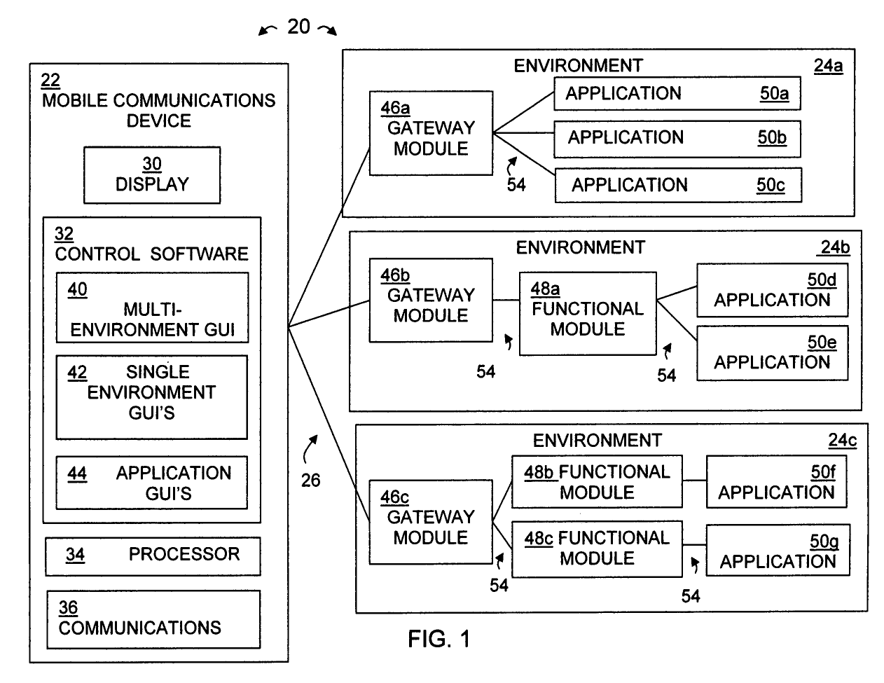

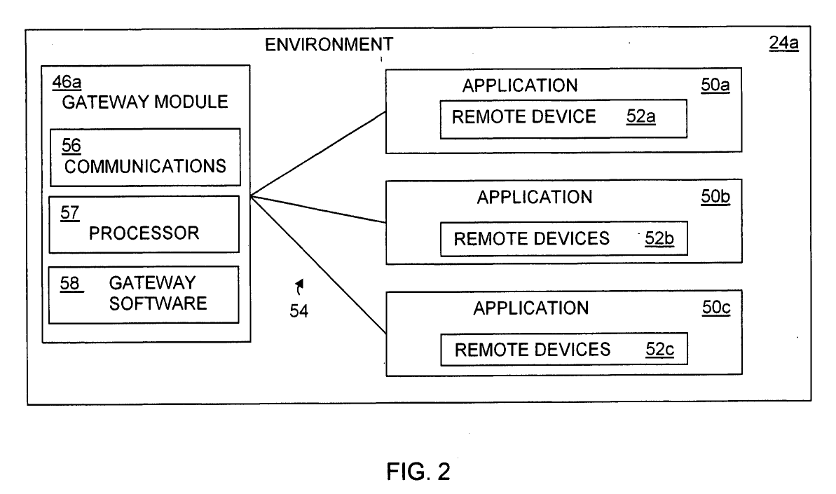

FIG. 1 depicts a multi-environment control and monitoring system (MCMS) 20 for controlling, monitoring, and using remote applications 50 according to the principles of the invention. Multiple instances of environments 24a, 24b, 24c, 24d, and 24e (in FIGS. 1,2,3,4, 7,) are described herein, also referred to generally as “environments 24”. Multiple instances of gateways 46a, 46b, 46c, 46d and 46e (in FIGS. 1,2,3,4, and 7) are described herein, also referred to generally as “gateways 46”. Multiple instances of functional modules 48a, 48b, 48c, (in FIGS. 1, 3, and 4) are described herein, also referred to generally as “functional modules 48”. Multiple instances of applications 50a, 50b, 50c, 50d, 50e, 50f, 50g, 50h, 50i (in FIGS. 1,2,3,4, and 7) are described herein, also referred to generally as “applications 50.” Multiple instances of remote devices 52a, 52b, 52c, 52d, 52e, 52f, 52g, 52h, 52i (in FIGS. 2, 3, and 4) are described herein, also referred to generally as “remote devices 52”.

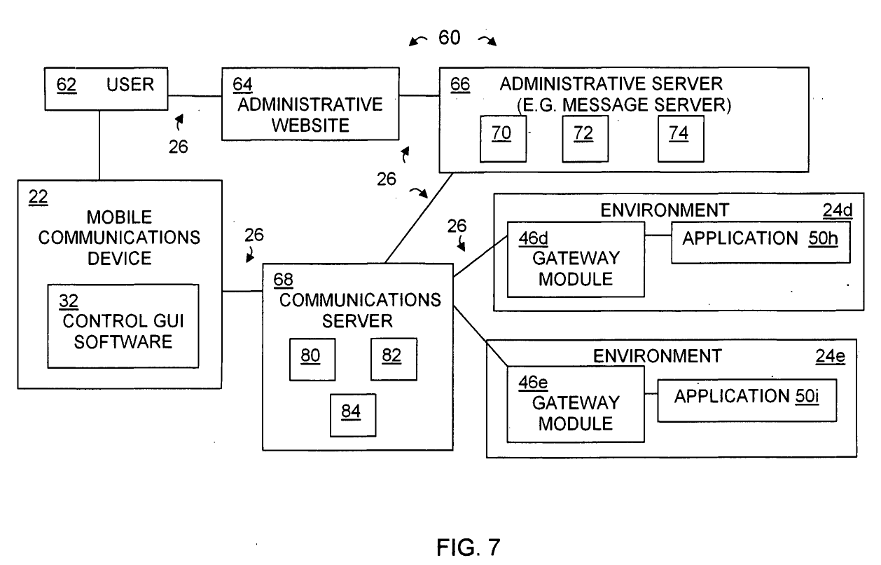

The system 20 includes the mobile communications device 22 (e.g., control device or cellular telephone) in communication with multiple gateways 46 (e.g., switchboards) to multiple environments 24, using communications connections 26, which communicate messages and/or data using one or more messaging protocols and/or communications protocols using point-to-point communications between a mobile communications device 22 and a gateway 46 and/or remote device 52. In an alternate embodiment, the mobile communications device 22 communicates with the gateway 46 (and/or remote device 52) through a server (e.g., administrative server 66, see FIG. 7). Each environment 24 includes one or more applications 50, which can include one or more remote devices 52, or one or more functional modules 48 that in turn control one or more remote devices 52 (see FIGS. 2, 3, and 4).

Each gateway 46 is in communication with each application 50 over environment communications 54 such as a local area network (LAN), or wireless connection. The mobile communications device 22 provides a GUI that presents a multi-environment menu 40 having icons for the multiple environments, single environment menus 42 having icons for applications within each single environment, and application submenus 44 having icons for application messages (e.g., commands and/or other options) for each application 50. The application messages can include macro commands, which include multiple application messages. The mobile communications device 22 provides one or more selected application messages to a gateway 46 for the selected environments 24, which receives each application message and acts as a switchboard to transfer each message to the appropriate application 50. The gateway 46 also receives informational messages (e.g., status, alarms) that are from remote devices 52 in response to one or more application messages or that are messages or alarms initiated by the application 50 and/or remote device 52, and transfers the messages to the appropriate mobile communications device 22.

The mobile communication device 22 is an electronic device capable of communicating with the gateway devices 46. The mobile communication device 22 can be a cellular telephone, portable short-range telephone, radio device, or other communications device. The mobile communications device 22 can also be a “hybrid” device including aspects of a computer and a communications device, such as a PDA (personal data assistant), wireless electronic mail device, laptop computer, palmtop computer, or other digital device with communication (e.g., wireless and/or cellular telephone) capabilities. The mobile communication device 22 can also be a hybrid or other electronic device to be developed with future technology that provides the functions of a mobile communication device 22.

The mobile communications device 22 includes a communications module 36 (e.g., cellular transceiver with antenna). The communications (e.g., communications connections 26) for the mobile communication device 22 are by means of wireless communications, such as cellular telephone communications, other radio wave communications, or other suitable electromagnetic wave communications. Suitable communications protocols for the communications connections 26 include CDMA (Code Division Multiple Access), iDEN (Integrated Dispatch Enhanced Network), GSM (Global System for Mobile Communications), GPRS (General Packet Radio Service), EvDO (Evolution Data Only), EDGE (Enhanced Data for GSM Evolution), 1xRTT, PDP (Packet Data Protocol), IEEE 802.11 (including 802.11a, 802.11b, and 802.11g), IEEE 802.15 (including 802.15.4), ZigBee, Bluetooth, wireless metropolitan area network (MAN) communications, WiMAX (IEEE 802.16 including 802.16d and 802.16e), Insteon™, satellite (e.g., in a low pass or geostationary orbit) communications, X10, Power Line, cable connections, and other suitable protocols and technologies. Alternatively, the communications connections 26 are by telecommunications and line connections, network connections, the Internet (TCP/IP or UDP/IP protocols), WAP (Wireless Application Protocol), or other suitable connections. For example, the mobile communication device 22 is a portable electronic device that can be plugged into network connections (e.g., communications connections 26), such as the Internet to provide communications with the gateway devices 46. More generally, the communications (e.g., communications connections 26) can be based on a combination of communication, messaging, connection, and/or networking technologies and protocols.

The communications connections 26 also rely on a messaging protocol, such as an SMS (short message service) or other suitable alphanumeric based messaging protocols. Messaging protocols also include those based on EMS (Enterprise Messaging Server), MMS (Multi-Media Messaging Service), IP (Internet Protocol), IMS (IP Multimedia Subsystem), and other suitable messaging protocols. Furthermore, the communications 26 of control commands and information can be based on high level alphanumeric and other communications protocols other than messaging protocols, such as Internet (TCP/IP or UDP/IP), mark-up language (e.g., HTML, XML), and other suitable protocols. Alternatively, the communications 26 rely on other communications protocols (e.g., data transfer protocols) capable of conveying messages, instructions and/or information between the mobile communications device 22 and one or more gateways 46, as well as communication protocols capable of performing the communications functions described herein that are developed in the future

The mobile communications device 22 includes a display 30, a processor 34 (e.g., digital microprocessor), and control software 32. The display 30 is an electronic display, such as an LCD display suitable for displaying graphic images, such as graphic icons. The instructions for the control software 32 are stored in a volatile memory (e.g., random access memory or RAM), and/or a nonvolatile memory (e.g., disk or nonvolatile memory IC (integrated circuit) chip). The processor 34 executes the instructions of the control software 32 to perform the functions of the control software 32 as described herein.

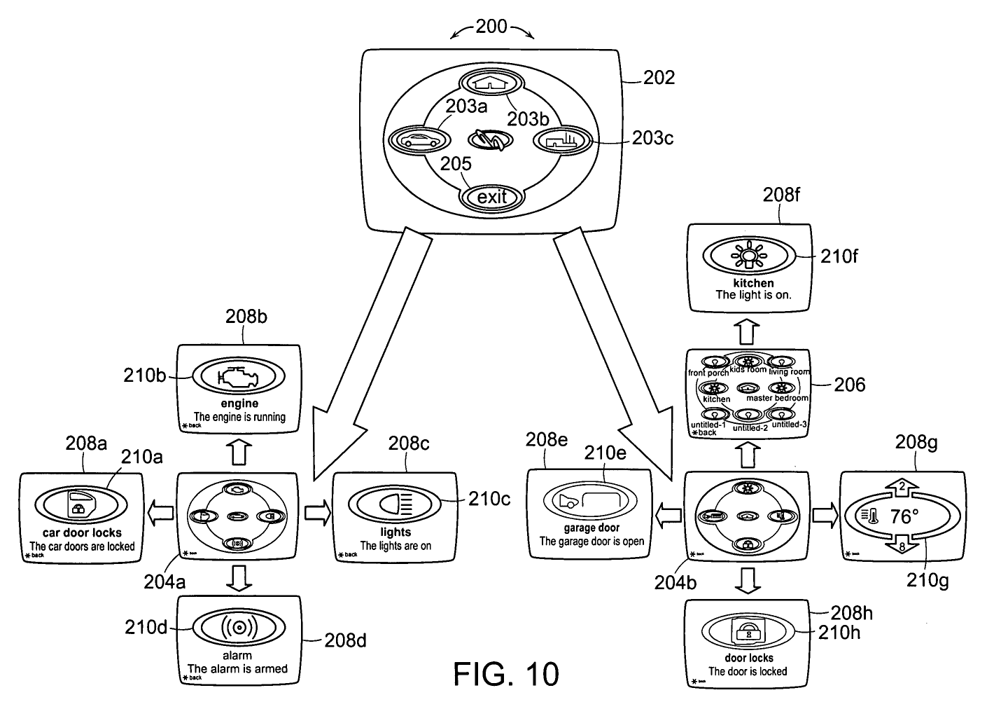

The control software 32 includes components for the GUI's, a multi-environment component that implements the multi-environment GUI 40, an environment component that implements the single environment GUI 42, and an application component that implements the application GUI 44. Each GUI software component of the control software 32 presents and manages a GUI 40, 42, 44 on the display 30 of the mobile communications device 22. The multi-environment GUI 40 includes, in one embodiment, a menu 202 (see FIG. 10) that allows a user 62 (see FIG. 7) to select an environment 24. The single environment GUI 42 includes, in one embodiment, a menu 204 (see FIGS. 10, 11, 12, 14, 15, and 29) that allows a user 62 to select an application 50 (or, in some cases, a remote device 52) within an environment 24. The application GUI 44 includes, in one embodiment, an application submenu 206 (see FIGS. 10, 14, and 15) that allows a user 62 to select a particular remote device 52 in an application 50. In other embodiments, the GUI control software 32 is developed based in Java J2ME, BREW, Macromedia Flash, Symbian, Windows Mobile, or other suitable development software and technologies, including development software and technologies to be developed in the future. The GUI control software 32 is adapted to the relatively small display size of most mobile communications devices 22 (e.g., cellular telephones), as well as usable on larger size displays (e.g., display for a laptop computer that has mobile communications facilities). In one embodiment, the functional module 48 has computing capabilities, or is a computer.

Each gateway module 46 includes a communications module 56 (e.g., transceiver, such as a cellular transceiver), a processor 57 (e.g., digital microprocessor), gateway software 58, and a communications port (e.g., antenna, such as a cellular antenna) (as shown, for example, for one gateway 46a in FIG. 2). The instructions 58 for the gateway module 46 are stored in a volatile memory, and/or a nonvolatile memory. The processor 57 in the gateway 46 executes the instructions of the gateway module software 58 to perform the functions of the gateway 46 as described herein. The functional module 48 is an electronics device that provides communications between the gateway 46 and one or more applications 50 (e.g., remote devices 52).

In an alternative embodiment, the processor 57 in the gateway 46 executes the instructions of the control software 32 (or instructions for components that implement one or more GUI's 40, 42, 44 of the control software 32) to perform the functions of the control software 32 (or one or more GUI's 40, 42, 44) as described herein. The gateway 46 and mobile communications device 22 exchange message and data transfers over the communications connections 26 to provide multi-environment GUI 40, single environment GUI's 42 and/or application GUI's 44 in the display 30 (e.g., see FIGS. 10, 11, 12, 13, 14, and 15). In one embodiment, the control software 32 uses WAP (Wireless Application Protocol) to communicate with the mobile communications device 22 using the communications connections 26 to provide the GUI's 40, 42, and/or 44. The approach of the invention is not limited to WAP, but other suitable wireless, GUI, networking protocols can be used to provide communications between control software 32 executing on a gateway 46 and the mobile communications device 22, including protocols and technologies to be developed in the future.

The communications 54 within the environment 24 are based on any suitable connection and networking or communications protocols, including local area network (LAN), wires or cables, wireless connections, infrared connections, optical connections, or any other suitable electromagnetic connections, including a combination of communications, connection, and/or networking technologies and protocols. The communications 54 in a home, building or facilities environment can include connections over the AC power lines or other existing wiring within the environment. Suitable wireless communications protocols include ZigBee, Bluetooth, IEEE 802.11 (including 802.11a, 802.11b, and 802.11g), IEEE 802.15 (including 802.15.4), wireless MAN protocols, WiMAX (IEEE 802.16 including 802.16d and 802.16e), Insteon™, iDEN (Integrated Dispatch Enhanced Network), CDMA (Code Division Multiple Access), GSM (Global System for Mobile Communications), GPRS (General Packet Radio Service), EvDO (Evolution Data Only), EDGE (Enhanced Data for GSM Evolution), 1xRTT, PDP (Packet Data Protocol), X10, Power Line, cable connections, and others suitable communications protocols, as well as protocols and communications technologies to be developed in the future. Alternatively, the environment communications 54 are based on any other suitable medium, such as mechanical or hydraulic connections (e.g., as can occur within an automobile).

Applications 50 are any device or system suitable for remote control and monitoring. An application 50 can include a single remote control device 52, such as a door lock on an automobile, or can be a system, such as a security system for a building, including multiple remote devices 52 such as door locks, video cameras, and other security devices.

FIG. 2 depicts an environment 24a including a gateway module 46a and applications 50a, 50b, and 50c according to the principles of the invention. In this embodiment of the invention, the gateway 46a communicates directly with the applications 50a, 50b, and 50c. Each application 50a, 50b, and 50c includes a remote device 52a, 52b, and 52c respectively. The gateway module 46a includes a communications module 56 (e.g., transceiver, such as a cellular transceiver), a processor 57 (e.g., digital microprocessor), gateway software 58, and a communications port (e.g., antenna, such as a cellular antenna), which can be included as components of any gateway module 46 described and/or illustrated herein.

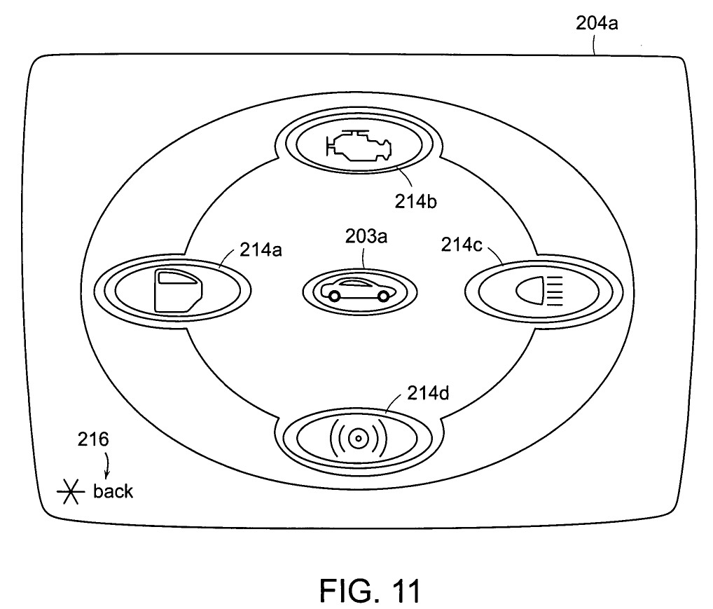

In one embodiment, environment 24a is an automotive environment accessed by selecting, in one embodiment, the automotive environment icon 203a from the multi-environment menu 202 (see FIG. 10) presented on the display 30 of the mobile communications device 22. The gateway 46a connects directly to the hard wired (or wireless) connections for the vehicle. The applications 50 include the door lock, ignition start, lights, security, and other applications. Thus the door lock application 50 includes a remote locking device for each door, which can be activated or deactivated from the applications submenu (e.g., 204a, FIGS. 10 and 11) or a lower level door lock submenu, if individual car doors are to be locked or unlocked) from the mobile communication device 22.

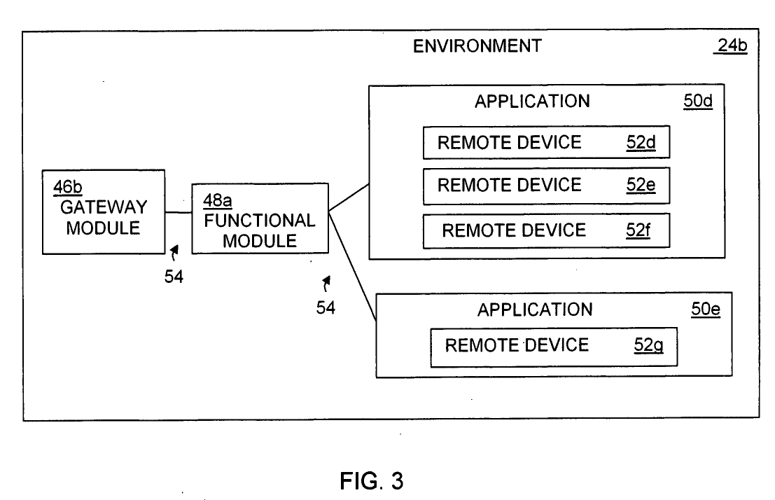

FIG. 3 depicts an environment 24b including a gateway module 46b, functional module 48a, and applications 50d and 50e according to the principles of the invention. In this embodiment of the invention, the gateway 46b communicates with a functional module 48a which in turn communicates with the applications 50d and 50e. Each application 50d and 50e includes one or more remote devices, shown as remote devices 52d, 52e, 52f for application 50d, and remote device 52g for application 50e.

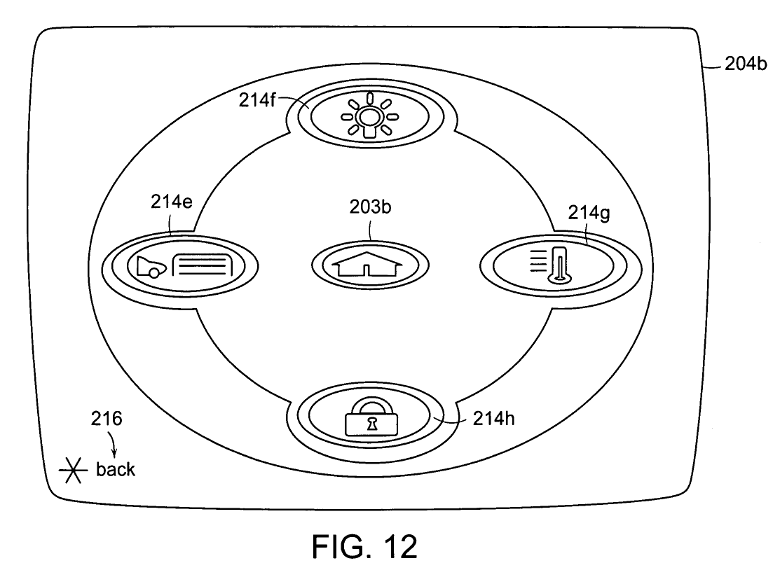

In one embodiment, environment 24b is a home environment accessed by selecting, in one embodiment, the home environment icon 203b from the multi-environment menu 202 (see FIG. 10) on the display 30 of the mobile communications device 22. The applications 50 can include door locks, switches (e.g., light switches), electrical outlets, climate controls, garage doors, kitchen appliances, security, and other applications. Thus the garage door can be activated from the garage door application submenu (e.g., 204b in FIGS. 10 and 12) displayed on the mobile communication device 22.

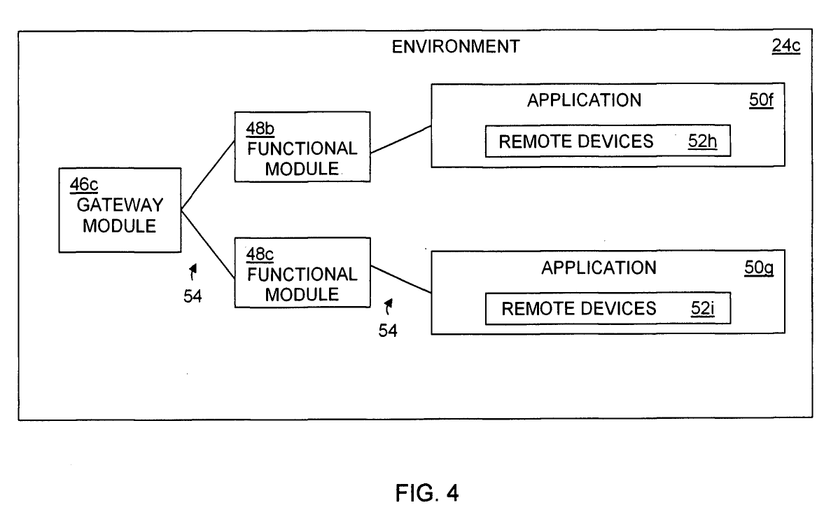

FIG. 4 depicts an environment 24c including a gateway module 46c, functional modules 48b, 48c, and applications 50f, 50g according to the principles of the invention. In this embodiment of the invention, the gateway 46c communicates with functional modules 48b, 48c which in turn communicate with the applications 50f, 50g. Each application 50f, 50g includes one or more remote devices 52h, 52i respectively. In one embodiment, the applications 52h, 52i are part of a remotely managed energy conservation system; for example, for facilities environments, including commercial, industrial, and institutional environments.

In one embodiment, environment 24c is a commercial facilities environment accessed, in one embodiment, by selecting a building environment icon 203c from the multi-environment menu 202 (see FIG. 10) on the display 30 of the mobile communications device 22. In this example, one functional module 48b controls the HVAC systems for the building, which can involve one or more applications 50 (e.g., 50f) with multiple remote devices 52h. The other functional module 48c controls the security systems for the building, which can involve one or more applications 50 (e.g., 50g) with multiple remote devices 52i.

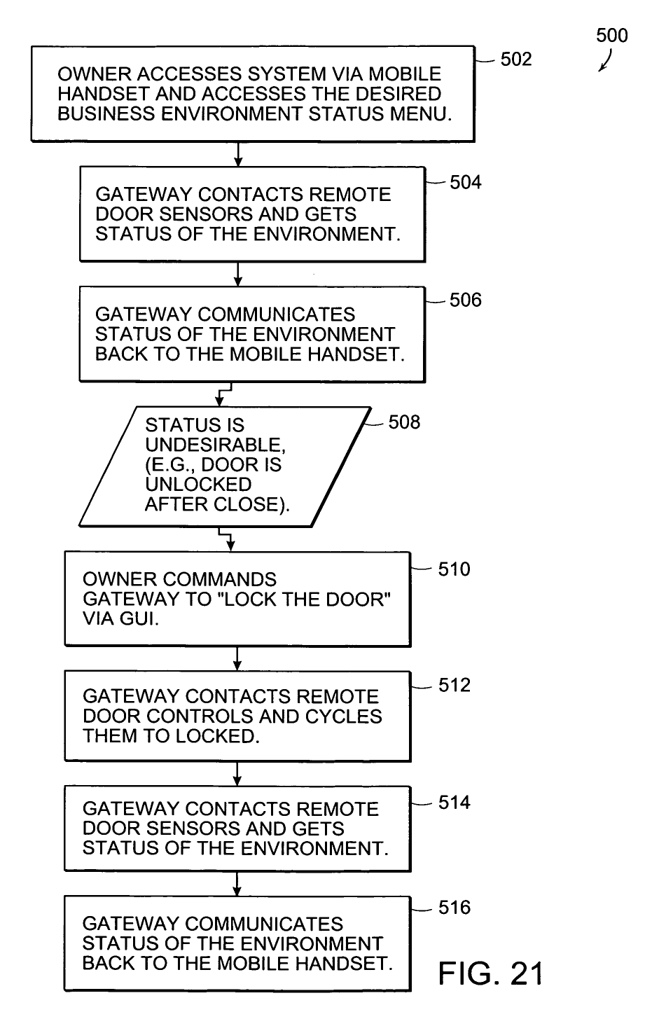

The control, monitoring and application use system 20 has other commercial applications. These commercial applications typically include one or more functional modules 48. The functional modules 58 for commercial applications can provide control of any electrical device or electrically controlled device 52 (which performs mechanical functions). Examples of these applications 50 are listed below (see also FIGS. 21-28), but the approach of the invention is not limited to this list:

Business Door/Entrance Control

Real Estate Door Lock and Unlock

Remote Water Meter Reading/Control

Remote Gas Meter Reading/Control

Medical Device Monitoring/Control—Diagnostics

Mechanical Control and Monitoring of Machinery

Rental Car Macro controls/Fleet Management

Fire Alarm and Monitoring/Control

Farm Irrigation Monitor and Control

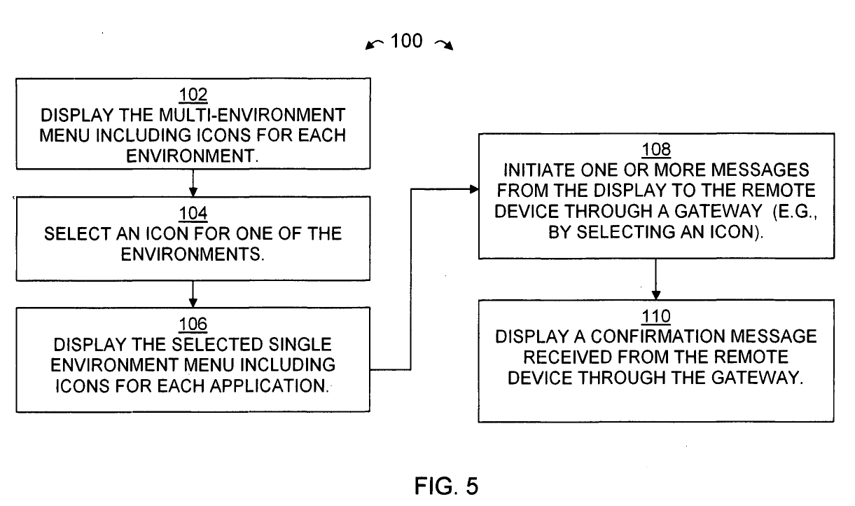

FIG. 5 depicts a procedure 100 for communicating with a remote device 52 from the display 30 of the mobile communications device 22, according to the principles of the invention, in one embodiment, using multi-environment menus 202 and environment menus 204 as shown in FIG. 10. The control software 32 presents (or provides) on the display 30 a multi-environment GUI 40 (step 102); for example, a multi-environment menu 202 including icons 203 for each environment 24. One environment 24 is selected by selecting one of the icons 203 in the multi-environment menu 202 (step 104). Typically, a user 62 of the mobile communications device 22 selects the icon 203. Alternatively, the selection is made automatically, such as by a software program executing on the processor 34 of the mobile communications device 22. The control software 32 includes single environment GUI software that presents (or provides) on the display an environment GUI 42 (step 106); for example, a selected single environment menu 204 including icons 214 (see FIGS. 11 and 12) for each application 50 in that environment 24. Then one or more messages are initiated by selecting an application icon 214 in the environment menu 204 for communication from the mobile communications device 22 in a point-to-point communication to the gateway 46 to the appropriate remote device 52 (step 108).

In one embodiment, a user 62 selects the automotive environment 24, and the control software 32 presents an automotive environment GUI 42; for example, a menu 204a of icons 214a, 214b, 214c, 214d, including an icon 214a for locking or unlocking the doors of a car in the automotive environment menu 204a (see FIG. 11). The user 62 then selects the door lock icon 214a, and the mobile communications device 22 sends a command to the gateway 46 located in the vehicle to unlock the car doors.

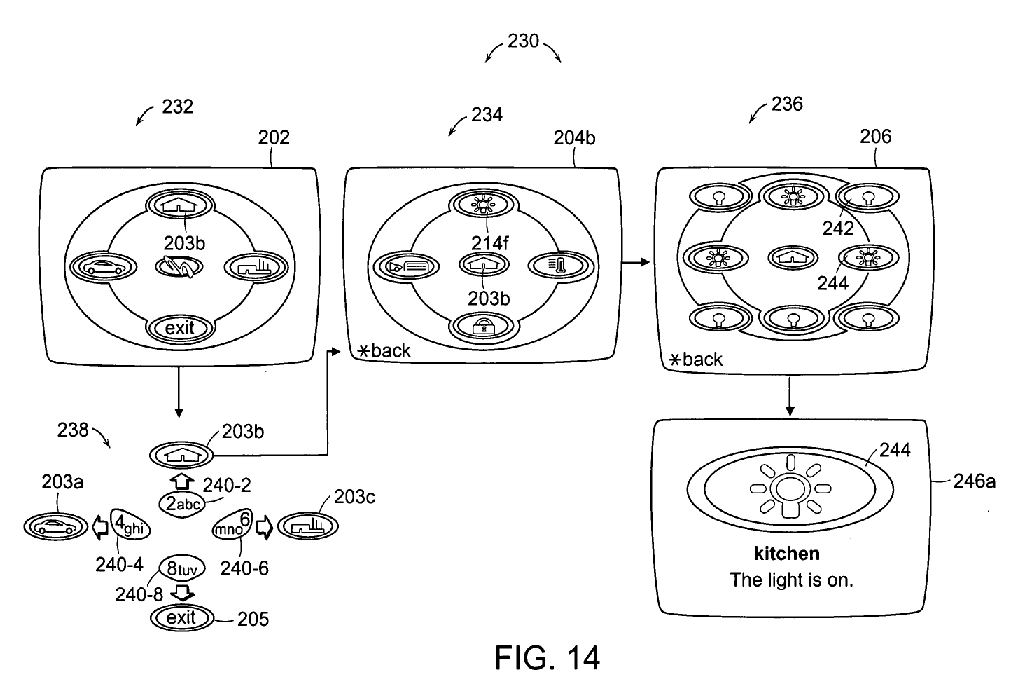

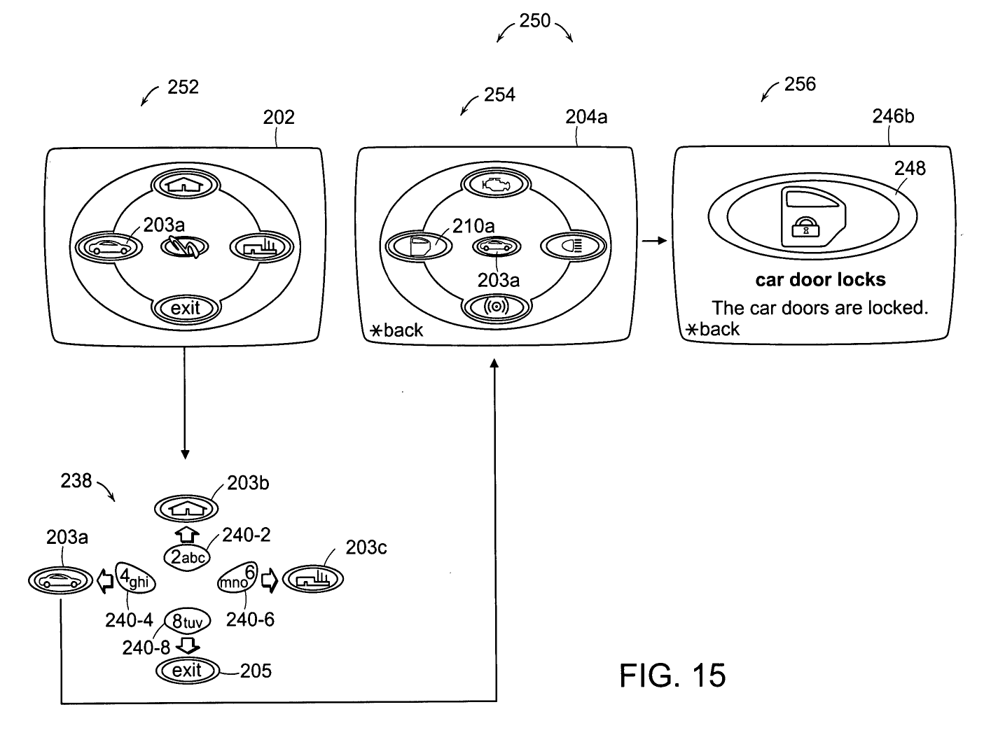

The mobile communications device 22 then presents (or provides) a confirmation message received from the remote device 52 through the gateway device 46 (step 110) such as a configuration screen 246 (see FIGS. 14 and 15).

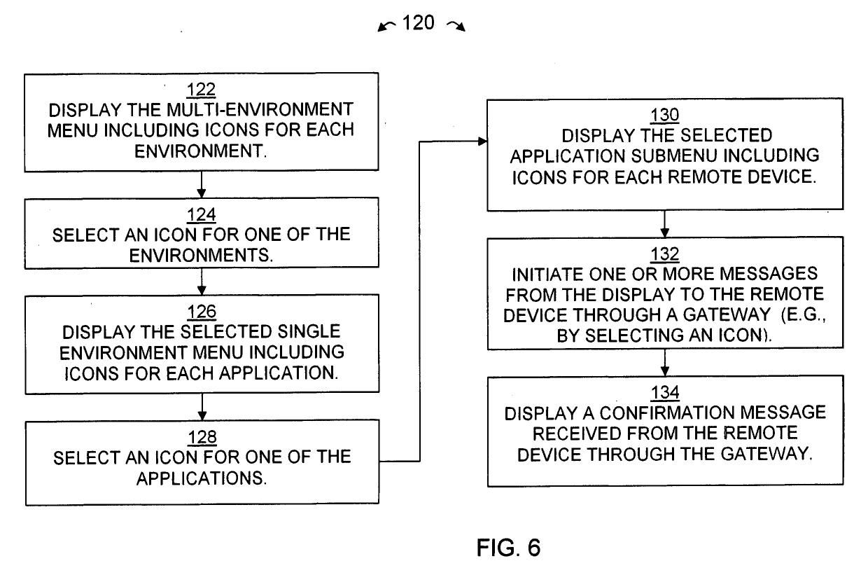

FIG. 6 depicts a procedure 120 for communicating with a remote device 52 from the display 30 of the mobile communications device 22 according to the principles of the invention, in one embodiment, using multi-environment menus 202, environment menus 204, and application submenus 206 as shown in FIGS. 10, 11, and 12. After an environment 24 is selected in procedure 120 (steps 122, 124), the mobile communications device 22 displays application icons 214, and the user 62 selects one of the application icons 214 (steps 126, 128). The mobile communications device 22 then displays a submenu 206 for the selected application icon 214 (step 130) (see FIG. 10), and the user 62 selects one of the icons (e.g., 242, 244) displayed in the submenu 206 (step 134) (see FIG. 14).

In one embodiment, the user 62 selects the lights icon 214f in the home environment 24, and then the control software 32 provides an application GUI 44; for example, an application submenu 206 showing the different lights in the home (see FIG. 14). The user 62 can then select the kitchen lights icon 244 to turn on the lights in the kitchen based on a point-to-point communication from the mobile communication device 22 to the appropriate gateway 46. The functional module 48 then returns a signal to the mobile communications device 22 through the gateway 46 to confirm that the kitchen lights 244 are on. The kitchen lights icon in the lights submenu 206 then changes appearance to indicate that the kitchen lights are on; for example, the kitchen light icon changes appearance from a dark light bulb (e.g., 242) to a white or bright light bulb (e.g., 244).

The mobile communications device 22 then presents (or provides) a confirmation message (e.g., confirmation screen 246a) received from the remote device 52 through the gateway device 46 (step 134).

FIG. 7 depicts a networked multi-environment administrative system 60 for managing remote applications 50 (e.g., controlling, monitoring and using the applications 50), according to the principles of the invention. The system 60 includes a mobile communication device 22, environments 24, gateways 46, and applications 50, an administrative web site 64, a administrative server 66, and a communications server 68. The mobile communications device 22 includes control software 32 (for example, including components implementing the multi-environment GUI 40, the single environment GUI 42, and the application GUI 44 of FIG. 1).

The administrative server 66 provides administrative services in connection with the control GUI software 32 and the control and monitoring services provided by the gateways 46. The administrative server 66 is also termed a “message server 66”. The administrative services include set up and configuration, registration, reports, and other services as described herein. The administrative web site 64 provides access to the administrative services for a user 62 of the system 60. Typically, the user 62 is also the user of the mobile communications device 22, although the user 62 of the web site 64 can also be an administrator or system administrator in a large organization or corporation where there are multiple users of mobile communications devices 22 and the control GUI software 32 included on the devices 22. The administrative web site 64 is hosted by the administrative server 66. Alternatively, the administrative web site 64 is hosted by a web server separate from the administrative server 66. In one embodiment, the administrative server 66 includes a communications module 70 (e.g., transceiver, such as a cellular transceiver), a processor 72 (e.g., digital microprocessor), administrative software 74, and a communications port (e.g., antenna, such as a cellular antenna). The instructions for the administrative software 74 are stored in a volatile memory, and/or a nonvolatile memory. The processor 72 in the administrative server 66 executes the instructions of the administrative software 74 to perform the functions of the administrative server 66 as described herein.

The administrative server 66 receives messages initiated by the mobile communications device 22, typically through a network aggregator that aggregates messages from different mobile communication devices 22 that can be using different mobile communications service providers (e.g., different cellular telephone service providers). Alternatively, the administrative server 66 receives the message directly through the communications connections 26.

The administrative server 66 authenticates that the message is from a mobile communications device 22 for a valid customer. In one embodiment, the administrative server 66 returns a revised version of the message to the mobile communications device 22 with an authentication or verification code, and the mobile communications device 22 sends the revised version of the message (e.g., remote control message) to the intended environment 24 (e.g., remote device 52 in the environment 24). In another embodiment, after authenticating the message, the administrative server 66 pairs the originating mobile communications device 22 to the intended environment 24 (e.g., to the intended gateway 46 and remote device 52 in the environment 24), and the administrative server 66 then transfers the message to the intended environment 24.

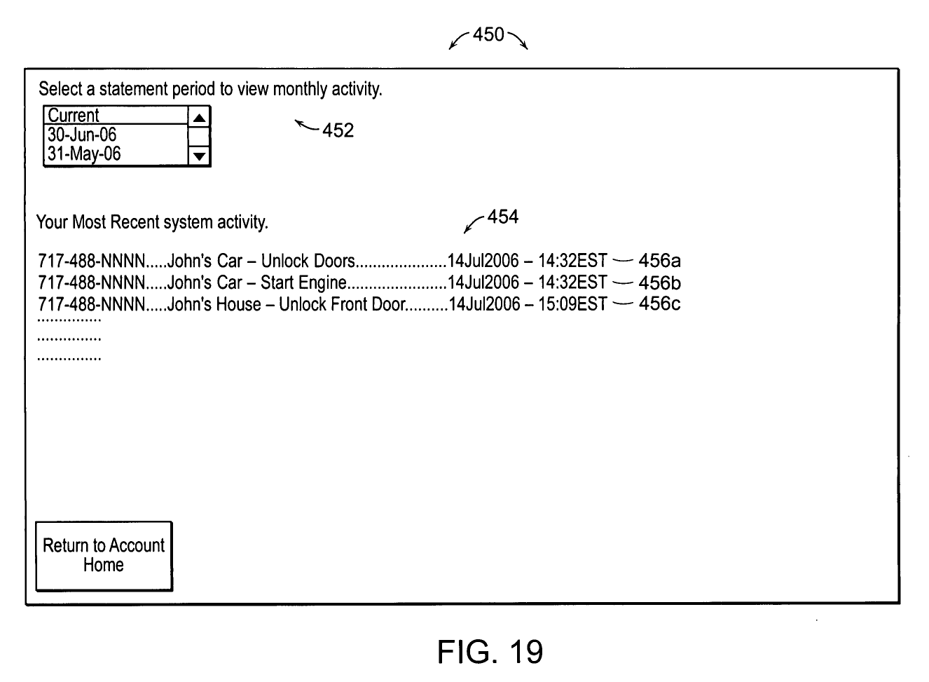

If an environment 24 (e.g., gateway 46 and/or remote device 52) initiates an alarm message, the administrative server 66 handles the message, performs authentication, and sends the message to the intended communications device 22. The server also includes or has access to a message data storage (volatile data storage and/or nonvolatile data storage, such as a hard disk) that provides storage for messages. The administrative server 66 can use the message data storage to produce an activity list 454 (see FIG. 19) based on messages initiated by a user 62, typically within a predetermined time period, such as predetermined by the system 20 (e.g., most recent activity) or selected by the user 62 (e.g., last week's or last month's activity) (see FIG. 19).

In one embodiment, the administrative server 66 receives an incoming message from the mobile communications device 22 in an SMS XML format (e.g., through a network aggregator). The format includes a destination address identifying the entity to receive the message, a source carrier address indicating the mobile communications service provider (e.g., used by the mobile communications device 22), and a data section that includes the message in a coded format. The decoded format includes a message with the following fields: prefix, message code, arg1, arg2, timestamp, and signature-hash. The following is a sample message: MOSHI:btnp;AAGGoQAAABA=;;20060417153450;tmh9Y+7vsalsjN+/DATtxA==:@

TABLE 1

| | |
|---|---|
| prefix | MOSHI |
| message code | btnp |
| arg1 | AAGGoQAAABA= |
| arg2 | (empty) |
| timestamp | 20060417153450 |
| sighash | tmh9Y+7vsalsjN+/DATtxA== |

The message code “btnp” indicates a button press action was taken at the GUI (e.g., environment GUI 42) presented on the display 30 of the mobile communications device 22. For example, the button press action selected a remote action for a remote device 52 in an environment 24 (e.g., door locking action for the doors of a remote vehicle). The arg1 field includes a component ID that is a global universal ID for a particular function for a particular environment 24 (e.g., identifies the door locking action for the doors of a particular remote vehicle). The signature-hash is a hash calculation used for authentication purposes

The message codes are as follows:

TABLE 2

| code | meaning | arg1 | arg2 |
|---|---|---|---|
| btnp | Button press | Component ID | (none) |
| btnr | Button response | Component ID | Result code |
| strq | Status request | Component ID | (none) |
| strp | Status response | Component ID | Status |
| mesg | Generic message | subject | body |
| pinr | Pin reset (from server) | (none) | (none) |
| alrm | Alarm notification | Component ID | Alarm text |

The result codes are as follows:

RESULT_ERROR=“error”;

RESULT_SUCCESS=“success”;

RESULT_UNKNOWN=“unknown”;

RESULT_ON=“on”;

RESULT_OFF=“off”;

In an alternative embodiment, the processor 72 in the administrative server 66 executes the instructions of the control software 32 (or instructions for one or more GUI's 40, 42, 44 of the control software 32) to perform the functions of the control software 32 (or one or more GUI's 40, 42, 44) as described herein. The administrative server 66 and mobile communications device 22 exchange message and data transfers over the communications connections 26 to provide multi-environment GUI 40, single environment GUI's 42 and/or application GUI's 44 in the display 30 of the mobile communications device 22 (e.g., see FIGS. 10, 11, 12, 13, 14, and 15). In one embodiment, the control software 32 uses WAP (Wireless Application Protocol) to communicate between the administrative server 66 and the mobile communications device 22 using the communications connections 26 to provide the GUI's 40, 42, 44. The approach of the invention is not limited to WAP, but other suitable wireless, GUI, and/or networking protocols can be used to provide communications between the control software 32 executing on the administrative server 66 and the displayed GUI's 40, 42, 44 of the mobile communications device 22, including protocols and technologies to be developed in the future.

The communications server 68 is an electronic or computer device (also termed a switch, cell switch, mobile switching center, network server, router, bridge, network appliance, or other suitable communications device) that provides switching and communications services among the mobile communications device 22, the administrative server 66, and the gateways 46 using the communications connections 26. These communications services can be based on mobile telephone protocols and technologies, other radio wave wireless technologies, or communications connections 26 as described elsewhere herein. In one embodiment, the communications server 68 includes a communications module 80 (e.g., transceiver, such as a cellular transceiver), a processor 82 (e.g., digital microprocessor), communications server software 84, and a communications port (e.g., antenna, such as a cellular antenna). The instructions for the communications server software 84 are stored in a volatile memory, and/or a nonvolatile memory. The processor 82 in the communications server 68 executes the instructions of the communications server software 84 to perform the functions of the communications server 68 (e.g., switching and routing of messages) as described herein.

In an alternative embodiment, the processor 82 in the communications server 68 executes the instructions of the control software 32 (or instructions for components that implement one or more GUI's 40, 42, 44) to perform the functions of the control software 32 (or one or more GUI's 40, 42, 44) as described herein. The communications server 68 and mobile communications device 22 exchange message and data transfers over the communications connections 26 to provide multi-environment GUI 40, single environment GUI's 42 and/or application GUI's 44 in the display 30 (e.g., see FIGS. 10, 11, 12, 13, 14, and 15). In one embodiment, the control software 32 uses WAP (Wireless Application Protocol) to communicate between the communications server 68 and the mobile communications device 22 using the communications connections 26 to provide the GUI's 40, 42, 44 to the display 30 of the mobile communications device 22. The approach of the invention is not limited to WAP, but other suitable wireless, GUI, networking protocols can be used to provide communications between the control software 32 executing on the communications server 68 and the displayed GUI's 40, 42, 44 mobile communications device 22, including protocols and technologies to be developed in the future.

In alternative embodiments, the processor 82 is implemented as multiple processors. The processor 82 includes one or more communications processors and a processor that executes the control software 32. In other embodiments, the communications server 68 is implemented as multiple communications servers 68 so that a message and/or data transfer exchanged between two or more of the mobile communications device 22, communications server 68, administrative server 66, and gateway 46 is transferred over the communications connections 26 through multiple communications servers 68.

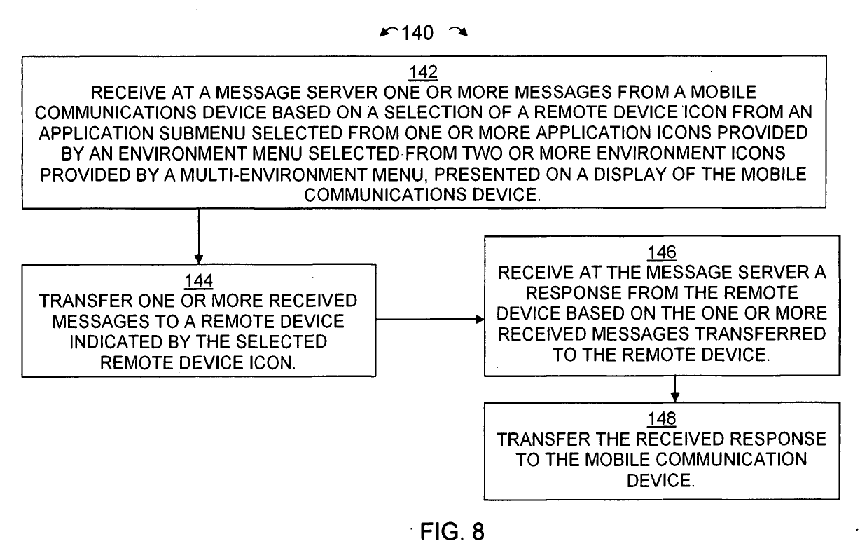

FIG. 8 is a procedure 140 for transferring multi-environment data (e.g., messages) through a message server 66 between a mobile communication device 22 and a remote device 52, according to the principles of the invention. The procedure 140 receives at a message server 66 data (e.g., one or more messages) from a mobile communications device 22 based on a selection of a remote device icon 210 from a selected application submenu 206 selected from one or more application icons 214 provided by an environment menu 204 selected from one or more environment icons 203 provided by a multi-environment menu 202, which have been presented on a display 30 of the mobile communications device 22 (see also FIGS. 1 and 10) (step 142). The procedure 140 transfers the received data (e.g., one or more received messages) to a remote device 52 indicated by the selected remote device icon 210 (step 144). The procedure 140 receives at the message server 66 a response based on the received data (e.g., one or more received messages) transferred to the remote device 52 (step 146). The procedure 140 transfers the received response to the mobile communication device 22 (step 148). In one embodiment, the data is alphanumeric data transferred based on the communications connections 26. In another embodiment, the data is a message in a message format or protocol.

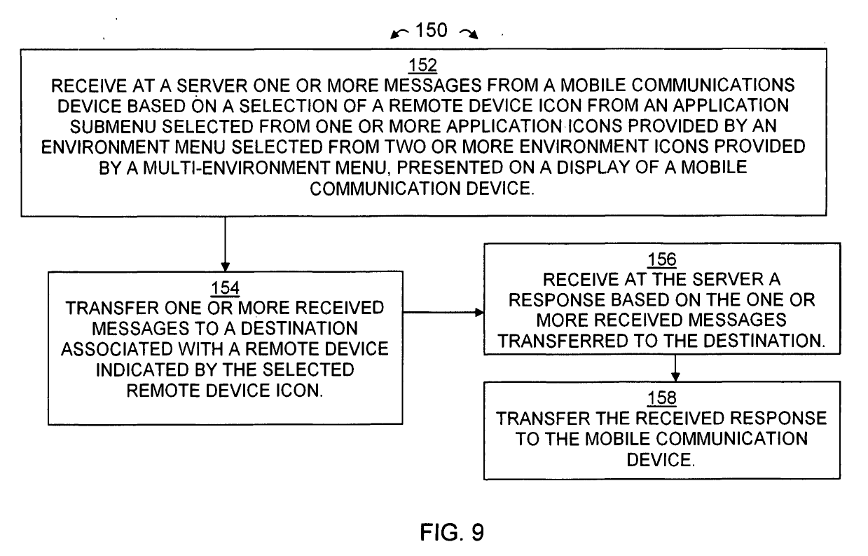

FIG. 9 is a procedure 150 for transferring multi-environment messages through a server (e.g., 66, 68) between a mobile communication device 22 and a destination, according to the principles of the invention. The procedure 150 receives at a server (e.g., administrative server 66 or communications server 68) one or more messages from a mobile communications device 22 based on a selection of a remote device icon 210 from a selected application submenu 206 selected from one or more application icons 214 provided by an environment menu 204 selected from one or more environment icons 203 provided by a multi-environment menu 202, which have been presented on a display 30 of the mobile communications device 22 (see also FIGS. 1 and 10) (step 152). The procedure 150 transfers one or more received messages to a destination associated with the selected remote device icon 210 (step 154). The procedure 150 receives at the server (e.g., 66 or 68) a response based on the one or more received messages transferred to the destination (step 156). The procedure 140 transfers the received response to the mobile communication device 22 (step 158). In various embodiments, the destination is a mobile communications device 22, a gateway 46, an application 50, a remote device 52, and/or the administrative server 66. In one embodiment, the data is alphanumeric data transferred based on the communications connections 26. In another embodiment, the data is a message in a message format or protocol.

FIG. 10 is a illustration of a GUI 200 for multiple environments, according to the principles of the invention. The multi-environment GUI 200 includes a multi-environment menu 202, environment menus 204a, 204b, (referred to generally as “environment menus 204”) and application submenu 206. The multi-environment menu 200 includes environment icons for an automotive environment 203a, a home environment 203b, and a facilities environment 203c, referred to generally as “environment icon 203,” and an exit icon 205. The automotive environment icon 203a can refer to any suitable vehicular environment 24 (e.g., car, truck, rental vehicle, ambulance, military vehicle, race car, or other suitable vehicle). Selecting the automotive environment icon 203a causes the display of the automotive environment menu 204a. The home environment icon 203b can refer to any suitable home environment 24 (e.g., primary residence, vacation residence, free-standing house, apartment, condominium, or other suitable home environment). Selecting the home environment icon 203b causes the display of the home environment menu 204b. The facilities environment icon 203c can refer to any suitable facilities (e.g., office building, commercial building, factory, warehouse, or other suitable facility). Selecting the facilities environment icon 203c causes the display of a facilities environment menu 204, (not shown in FIG. 10).

The multiple environment GUI 200 also includes remote device screens 208a, 208b, 208c, 208d, 208e, 208f, 208g, and 208h, referred to generally as “remote device screens 208,” and remote device icons 210a, 210b, 210c, 210d, 210e, 210f, 210g, and 210h, referred to generally as “remote device icons 210.” The automotive environment menu 204a provides access to a car lock screen 208a including a car door lock icon 210a, an start engine screen 208b including engine icon 210b, a car lights screen 208c including car lights icon 210c, and a car alarm screen 208d including car alarm icon 210d. The home environment menu 204b provides access to a garage door screen 208e including a garage door icon 210e, a lighting application submenu 206, a temperature control screen 208g including temperature control icon 210g, and door locks screen 208h including door locks icon 210h. The lighting application submenu 206 displays multiple icons for different lights in the home that provide access to a kitchen lights screen 208f including kitchen lights icon 210f.

The remote device screen 208 serves as a confirmation screen providing an indication of the current status of a remote device 52. For example, remote device screen 208a provides a confirmation that the car doors are locked, as indicated by the car doors locked icon 210a. In other embodiments, the remote device icon 210 displayed in the remote device screen 208 provides access to other submenus, such as an application submenu 206, or provides further actions. In one embodiment, the remote device icon 210 can also provide access to a remote device submenu that presents icons for one or more remote actions. For example, the kitchen lights screen 208f indicates that the kitchen light is on with a default setting of medium light intensity, and selecting the kitchen lights icon 210f provides access to a remote device submenu for adjusting the intensity of the light. For example, the submenu provides remote actions for the intensity of the light, such as for a 3-way light having icons for a low intensity remote action, a medium intensity remote action, and a high intensity remote action.

The approach of the invention is not restricted to the menus 202, 204 and submenus 206 shown in FIG. 10 and/or described herein, but the menus 202, 204, submenus 206, and icons 210, 214 can be combined and arranged in various ways to support the approach of the invention as described herein; providing a user 62 the ability to control and monitor remote devices 52 in various environments 24.

FIG. 11 is an illustration of the automotive environment menu 204a of FIG. 10, according to the principles of the invention. FIG. 12 is an illustration of the home environment menu 204b of FIG. 10, according to the principles of the invention. FIGS. 11 and 12 illustrate application icons 214a, 214b, 214c, 214d, 214e, 214f, 214g, and 214h, referred to generally as “application icons 214.” The environment menus 204a, 204b also include a back icon 216 that allows the user 62 to return to the previous menu (e.g., the multi-environment menu 202). The automotive environment menu 204a includes a car door application icon 214a, engine application icon 214b, car lights application icon 214c, and car alarm application icon 214d. The home environment menu 204b includes a garage door application icon 214e, lights application icon 214f, temperature control application icon 214g, and door locks application icon 214h. If the application 50 has one remote device 52, then the selection of an application icon 214 leads to the display of a remote device screen 208, which provides a remote device icon 210. In this case, the application icon 214 may have a similar appearance to the remote device icon 210. For example, the selection of a car door application icons 214a (FIG. 11) leads to the display of a car door locked screen 208a (FIG. 10), which provides a locked car door icon 210a. The car door application icon 214a has a similar (but not identical) appearance to the car door locks icon 210a. However, the approach of the invention does not require that menu selections (e.g., car door application icon 214) have an identical appearance to a related icon in a lower level menu (e.g., locked car door lock 210a).

The approach of the invention does not require a particular configuration for icons (e.g., 203, and 214) in a graphic menu (e.g., 202, 204), such as shown in FIGS. 10, 11, and 12. The approach of the invention also supports other configurations, such as those based on horizontal rows, vertical columns, diagonals, geometric figures (e.g., triangle, pentagon, hexagon, etc.), and other suitable configurations. The approach of the invention also supports various designs for icons (e.g., 203, 210, 214) other than the oval shape shown in FIGS. 10, 11, and 12; for example, supporting, circular, rectangular, triangular, and other suitable geometric shapes.

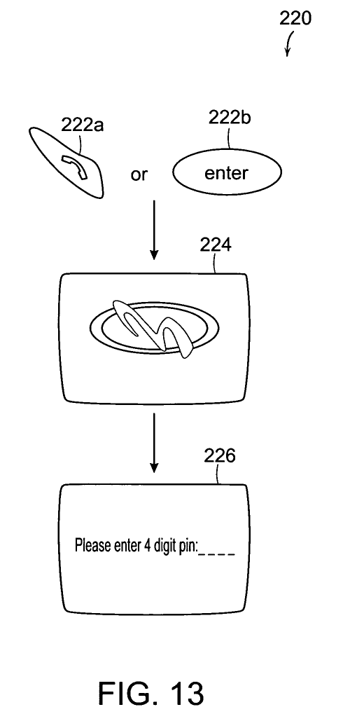

FIG. 13 is a diagram that depicts an access procedure 220 for gaining access to the multi-environment control and monitoring system 20 for the multiple environments GUI 200 of FIG. 10, according to the principles of the invention. The user 62 presses a key 222a or 222b, (referred to generally as “enter key 222” or “access key 222”) on the mobile communications device 22 to initiate access to the MCMS system 20. In various embodiments, the user 62 initiates access by using any of a number of suitable approaches, such as speaking a voice prompt from the user 62, pressing a specific sequence of alphanumeric keys on the keypad 238 of the mobile communications device 22, or other approaches. The system 20 presents a logo screen 224 in the display 30 of the mobile communications device 22 and sounds a tone (e.g., chime or other tone) to indicate the initiation of the MCMS system 20. In various embodiments, various graphics and/or sounds can be used to indicate initiation of the MCMS system 20. The MCMS system 20 then provides an on-screen message on the display 30 of the mobile communications device 22 providing a security feature, such as the request for a PIN (personal identification number) shown in the security screen 226. In various embodiments, various security features can be used, such as biometric security (e.g., thumbprint or other fingerprint pad associated with the mobile communications device 22 or voiceprint), or other suitable security feature. After the user 62 has successfully entered the security data (e.g., PIN), the MCMS 20 then allows access to the main environment menu 202 of the MCMS 20 (FIGS. 14 and 15). In one embodiment, the secure access is provided as a function of the control software 32. In other embodiments, the security function is provided by a separate security module that is not part of the control software 32, and can be executed elsewhere (e.g., at the administrative server 66, the communications server 68, and/or a security server).

FIG. 14 is a diagram that depicts a lighting control procedure 230 for accessing a lighting application submenu 206 for the multiple environments GUI 200 of FIG. 10, according to the principles of the invention. After obtaining secure access to the MCMS system 20, for example by entering a PIN (see FIG. 13), the user 62 is directed to the multi-environment menu 202 (step 232). The selections displayed on the display 30 of the mobile communications device 22 are mapped to navigation controls, such as keys 240-2, 240-4, 240-6, and 240-8 (referred to generally as “navigation keys 240”), shown, for example, in FIG. 14 as the 2, 4, 6, and 8 keys of the keypad 238 of the mobile communication device 22. The keypad 238 normally contains other numeric keys and other kinds of keys (e.g., the enter keys 222 of FIG. 13). In another embodiment, the navigation keys 240 are implemented as arrow keys, for up, down, right, and left. In the example shown in FIG. 14, the navigation key 240-2 maps to the home environment icon 203b, the navigation key 240-4 maps to the automotive environment icon 203a, the navigation key 240-6 maps to the facilities environment icon 203c, and the navigation key 240-8 maps to an exit key 205. The user 62 desires to turn a light on or off in the kitchen from his workplace. So first, the user 62 operates (e.g., presses) the navigation control, such as key 240-2, to highlight the home environment icon 203b. Then the user 62 operates (e.g., presses) a selection control, such as a selection key (e.g., 222) to select the home environment icon 203b and to indicate his desire to access the home environment menu 204b. In other embodiments, the selection is made in alternative modes, such as operating (e.g., pressing) an “OK” key on the mobile communications device 22 as the selection control, pressing the appropriate navigation key 240 twice, operating some other selection control, such as a selection control on the side of a cellular telephone, speaking “OK” or other appropriate voice prompt into the telephone, or other suitable selection mechanism.

The user 62 is then directed to the home environment menu 204b (step 234). The home environment icon 203b is displayed in the center of the home environment menu 204b and lets the user 62 know that the user 62 is currently under the “home” menu 204b. The user 62 operates (e.g., presses) the navigation control, such as key 240-2, to access the lighting multiple icon application submenu 206, because the key 240-2 is mapped to this submenu 206 in step 234 (which is different from the mapping of the keys 240 in step 232).

The user 62 is then directed to the lighting application submenu 206 (step 236). This submenu 206 displays all of the lights that the user 62 can control. To turn a light on, the user 62 operates (e.g., presses) the corresponding navigation control, such as key 240, for the light that they desire to turn on or off. So, in the example in FIG. 14, the user 62 presses the 240-6 navigation key, which switches the light on or off. The light off condition is indicated by a dark light icon (e.g., 242), and the light on condition is indicated by a bright light icon (e.g., 244). A lights confirmation screen 246a briefly appears in the display 30 of the mobile communications device 22 and informs the user 62 that the light has been turned on or off; for example, in FIG. 14, the confirmation screen 246a indicates that the kitchen light has been turned on as indicated by the bright light icon 244. After a few seconds, the user 62 is returned to the lights control menu 206, where he can continue to make further changes. Confirmation screens 246a (FIG. 14) and 246b (FIG. 15) are referred to generally as “confirmation screens 246.”

FIG. 15 is a diagram that depicts a car door locks procedure 250 for accessing a car door locks application 50 for the multiple environments GUI 200 of FIG. 10, according to the principles of the invention. After obtaining secure access to the MCMS system 20, for example by entering a PIN (see FIG. 13), the user 62 is directed to the multi-environment menu 202 (step 252). The selections displayed on the display 30 of the mobile communications device 22 are mapped to navigation keys 240-2, 240-4, 240-6, and 240-8 (referred to generally as “navigation keys 240”), as described for FIG. 14. The user 62 desires to lock or unlock his car doors from his office. So first, the user 62 operates (e.g., presses) the navigation control, such as key 240-4 and a selection control (e.g., selection key 222), as described for FIG. 14, to indicate a desire to access the automotive environment menu 204a.

The user 62 is then directed to the automotive environment menu 204a (step 254). The automotive environment icon 203a displayed in the center of the automotive environment menu 204a lets the user 62 know that the user 62 is currently under the “automotive” menu 204a. The user 62 operates (e.g., presses) the navigation control, such as key 240-4, to lock or unlock the car doors.

A car door locks confirmation screen 246b briefly appears in the display 30 of the mobile communications device 22 and informs the user 62 that the car doors have been locked or unlocked; for example, in FIG. 15, the confirmation screen 246b indicates that the car door locks have been locked as indicated by the locked car door icon 248. After a few seconds, the user 62 is returned to the automotive environment menu 204a, where he can continue to make further changes.

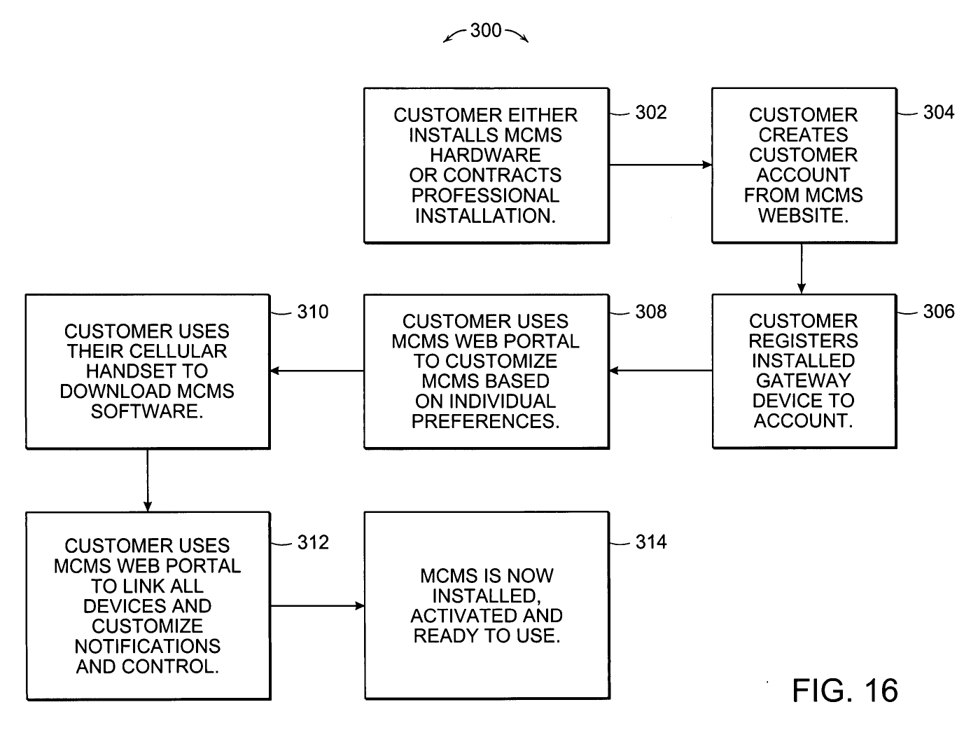

FIG. 16 is a flowchart that depicts an installation procedure 300 for registering and installing the MCMS control software 32. The user 62 (e.g., customer) either installs the MCMS hardware (e.g., gateway module 46) or contracts for professional installation (step 302). For example, the customer or professional installer installs a gateway 46 for a home environment 24 that provides for the control and monitoring of remote devices 52, such as a garage door, lights, door locks, temperature control and/or other suitable remote devices 52. The customer creates a customer account by accessing the MCMS administrative web site 64, for example, by providing name, address, electronic mail address, and other relevant data, and by creating a Web user ID and Web PIN (step 304). The customer registers the installed gateway 46 to the customer account (step 306). The customer uses the MCMS web portal 64 to customize the MCMS system 20 based on individual preferences (step 308). The customer can choose, for example, to have a custom name and icon for different environments and applications. For example, if the customer has two cars, unique names and icons can be assigned to each car (e.g., “SUV” and “coupe”). For example, if the customer has two residences, they can assign unique names and icons to each house (e.g., “home” and “vacation cottage”).

The customer uses the mobile communications device 22 (owned or controlled by the customer) to download the MCMS control software 32 (step 310) to the mobile communications device 22. In one embodiment, the customer establishes a connection between the mobile communications device and control software 32 that resides on an administrative server 66, communications server 68, and/or a gateway 46. In another embodiment, the customer downloads software (e.g., client, applet, distributed object, or other software) to the mobile communications device 22 that accesses the control software 32 which resides as software (e.g., server, web services, distributed object, servlet, or other software) on an administrative server 66, communications server 68, and/or a gateway 46.

The customer uses the MCMS web portal 64 to link all devices (e.g., gateways 46 and remote devices 52) and customize notifications and control (step 312). The MCMS system 20 and control software 32 is now installed, activated, and read to use (step 314).

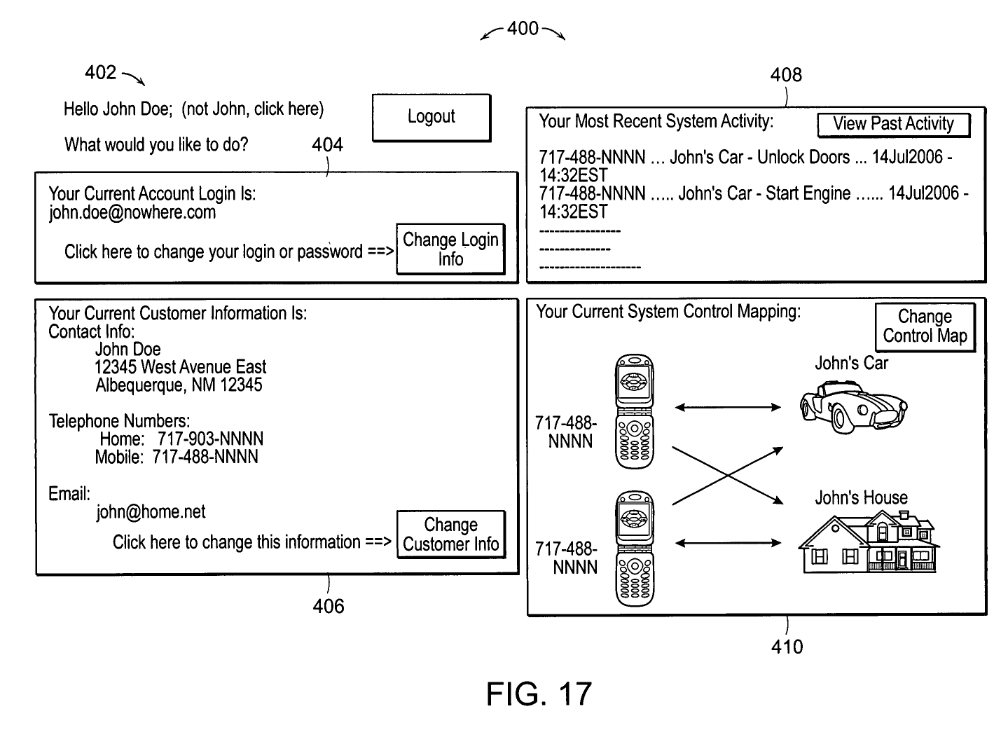

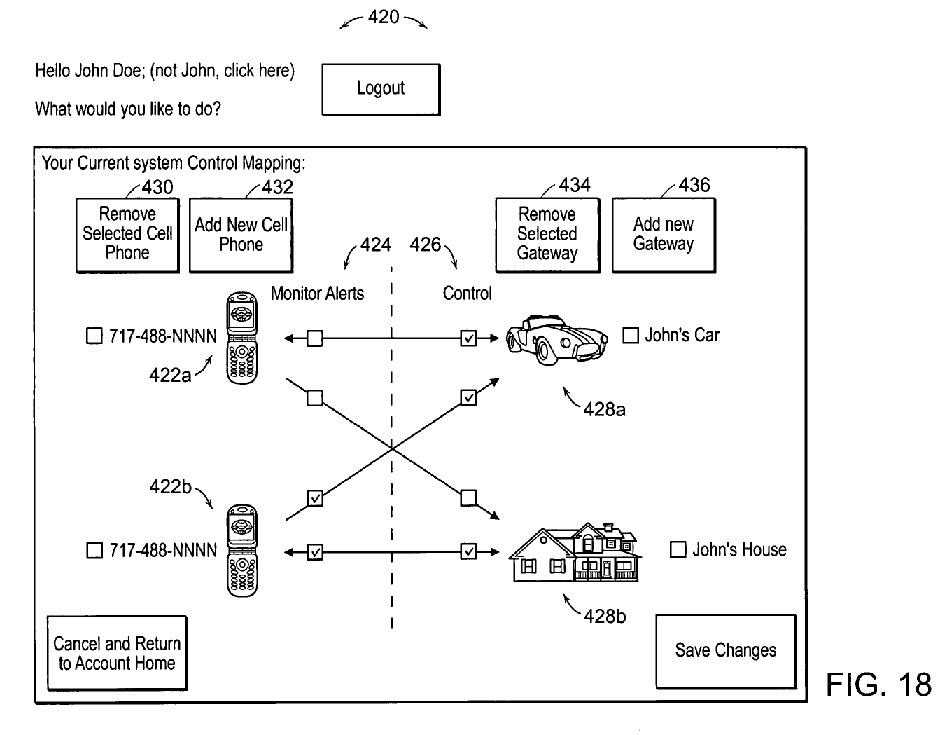

FIG. 17 is an illustration of a customer account home screen 400, according to the principles of the invention. The display screen 400 (as well as display screens 420, 450, and 470) is displayed on the administrative web site 64 for the user 62 and is accessed after the user 62 (e.g., customer) completes a security screen (e.g., by entering a password) (see FIG. 13). The display screen 400 includes a salutation section 402, login section 404, customer information section 406, system activity section 408, and control mapping section 410. The salutation section 402 presents a greeting to the user 62 as well as other information, and asks the user 62 “what would you like to do?” which indicates that the user 62 is invited to select an action based on one of the displayed sections of information 404, 406, 408, 410. The login section 404 provides the user 62 with the option of changing the login information and password. The customer information section 406 presents the customer information and provides the user 62 with the option to change the customer information. The system activity section 408 presents the most recent activity and indicates that the customer can view past activity (see FIG. 19, which shows a more complete listing of recent activity). The control mapping section 410 presents a control mapping for two mobile communications devices 22 (e.g., two mobile telephones), and two environments (e.g., John's Car and John's House), as discussed further for FIG. 18). In other embodiments, the customer account home screen 400 includes various sections of information and combinations of sections that differ from those shown in FIG. 17, related to various aspects of the invention as disclosed herein.

FIG. 18 is an illustration of a change control mapping screen 420 related to the customer account home screen 400 of FIG. 17, according to the principles of the invention. The change control mapping screen 420 include mobile communications device information 422 (e.g., 422a and 422b), monitor alert check boxes 424, control check boxes 426, remote gateway graphics 428 (e.g., 428a and 428b), a remote selected cell phone button 430, an add new cell phone button 432, a remove selected gateway button 434, and add new gateway button 436. The mobile device information 422 includes information about one or more mobile communications devices 22 that are owned and/or controlled by the user 62, including the mobile communications device telephone number or other identifier as part of the information 422. The remote gateway graphics 428 provide information for one or more gateways 46 and corresponding environments 24 that can be accessed by the user 62, such as a remote gateway graphic 428a for a car owned and/or controlled by the user 62 (e.g., “John's Car”) and a remote gateway graphic 428b for a house owned and/or controlled by the user 62 (e.g., “John's House”).

The user 62 can check off one or more monitor alert boxes 424 and one or more control boxes 426 to set up levels of monitoring and control of the gateways 46 associated with the remote gateway graphics 428. For example, the user 62 chooses for the mobile communications device 22 indicated by 422a to control the car gateway 46 indicated by remote gateway graphic 428a as indicated by a checked off control box under control boxes 426 but not to receive alerts as indicated by no check-off under the monitor alert checkboxes 424 next to 422a. Also the user 62 chooses for the mobile communications device 22 indicated by 422a to neither control nor monitor the house gateway 46 indicated by remote gateway graphic 428b. The user 62 chooses for the mobile communications device 22 indicated by 422b to control and monitor the car gateway 46 indicated by remote gateway graphic 428a, and to control and monitor the house gateway 46 indicated by remote gateway graphic 428b. The user 62 presses the buttons 430 or 432 to remove or add a selected cell phone (that is, any suitable mobile communications device 22) using the checkboxes associated with the telephone numbers for the mobile device information 422a and 422b. The user 62 presses the buttons 434 or 436 to remove or add a selected gateway 46, using the checkboxes associated with the remote gateway graphics 428a and 428b.

FIG. 19 is an illustration of an account activity screen 450 related to the customer account home screen 400 of FIG. 17, according to the principles of the invention. The account activity screen 450 provides a select time period section 452 to view activity on a monthly basis. In other embodiments, the user 62 selects other time periods (e.g., daily, weekly, quarterly, annually, and so on) as desired. The account activity screen 450 provides a listing of the system activity 454, which includes activity to unlock car doors 456a, activity to start a car engine 456b, and activity to unlock a house front door 456c (referred to generally as “activities 456”). Thus the account activity screen 450 combines activities 456 for various gateways 46 and environments 24. In other embodiments, the system activity organizes the activities 456 by gateway 46, time period, originating mobile communications device 22, and/or other suitable criteria based on aspects of the invention as described herein.

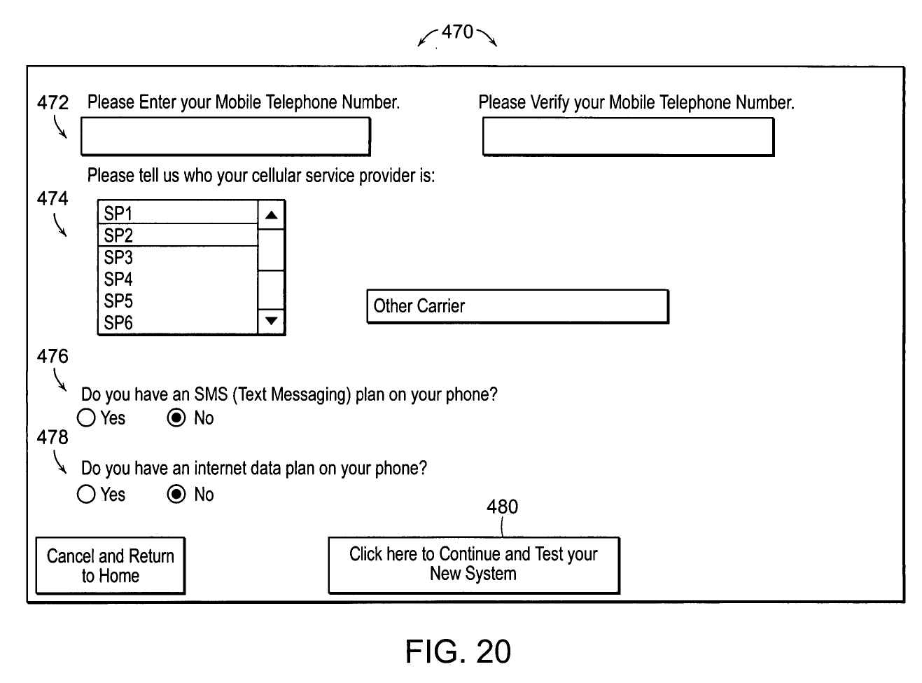

FIG. 20 is an illustration of a software download screen 470 related to the customer account home screen 400 of FIG. 17, according to the principles of the invention. The software download screen 470 provides a mobile identifier data entry section 472, a service provider (SP) data entry section 474, a confirm message support section 476, a confirm internet data support section 478, and a software download button 480. In other embodiments, software download screen 470 is not required to include all of these sections 472, 474, 476, 478, and other sections and buttons can be included based on aspects of the invention as described herein. The mobile identifier data entry section 472 asks for the user's 62 mobile communication device 22 identifier (e.g., telephone number). The service provider data entry section 474 asks the user 62 to identify the service provider (e.g., cellular telephone service provider) for the mobile communications device 22. The confirm message support section 476 asks the user 62 to confirm (or not confirm) message support (e.g., SMS support) for the mobile communications device 22. The confirm internet data support section 478 asks the user 62 to confirm (or not confirm) internet data support for the mobile communications device 22. After providing the data requested in sections 472, 474, 476, and 478, the user 62 presses the software download button 480 to continue the software installation process, initiate download of the control software 32 to the mobile communications device 22, and test the installation of the control software 32. In other embodiments, pressing the software download button 480 initiates the establishment of a connection between the mobile communications device 22 (e.g., downloading of a client, applet, distributed object, or other local software to the mobile communications device 22) and remote control software 32 that resides elsewhere (e.g., is stored and executed on a gateway 46, administrative server 66, and/or communications server 68).

FIG. 21 is a flowchart that depicts a procedure 500 for business door control in a business facilities environment 24, according to the principles of the invention. In this procedure 500, a small business owner (e.g., the user 62) wants to verify the status of a door after an employee closes the shop. The owner accesses the system 20 via mobile handset (e.g., system enabled mobile communications device 22) and accesses the multi-environment GUI 40 to display the desired environment status menu (e.g., environment GUI 42 or application GUI 44); such as accessing a business environment menu from an environment menu 42 (step 502). For example, business status environment menus show door lock status for one or more locks at the shop). In alternate embodiments, the business environment status menu is the same as the environment menu 42, or is a multiple icon application submenu 46. In one embodiment, a multi-environment GUI 40 also includes other environment menus 42, such as for the owner's home, business vehicle, and other suitable environments 24.

The appropriate gateway 46 at the business facilities environment 24 (e.g., small business shop) contacts the remote application 50 (e.g., including door sensors) and gets status of the remote device 52 (e.g., door locks) in the environment 24 (step 504). The gateway 46 communicates status of the environment 24 back to the control software 32 for presentation on the display 30 of the mobile communications device 22 (e.g., mobile handset) (step 506). The status is undesirable, (e.g., door is unlocked after close) (step 508). The owner (e.g., user 62) commands the gateway 46 to “lock the door” by entering the command via the GUI (e.g., application submenu GUI 44) presented by the control software 32 on the display 30 (step 510). The gateway 46 contacts the remote application 50 (e.g., remote door controls) in the business facilities environment 24 and cycles the doors to be locked (step 512). The gateway 46 contacts the remote application 50 (e.g., including door sensors) and gets status of the remote device 52 (e.g., door locks) in the business facilities environment 24 (step 514). The gateway 46 communicates status of the environment 24 back to the control software 32 to present the status (e.g., locked status) in the display 30 at the mobile communications device 22 (e.g., mobile handset).

A process similar to this procedure 500 can be used to unlock the door in the event that an employee has forgotten the key. For example, the owner (e.g., user 62) commands the gateway 46 to unlock the remote device 52 (e.g., door locks) in step 510, and steps 512, 514, and 516 are modified accordingly, so that the control software 32 presents an unlocked status in the display 30 at the mobile communications device 22 (e.g., mobile handset).

In other embodiments, variations of the procedure 500 are used to accomplish other tasks appropriate for the business facilities environment 24 (e.g., shop or other business environment), such as checking and adjusting temperature, checking and adjusting lights, inventory management, and other suitable tasks.

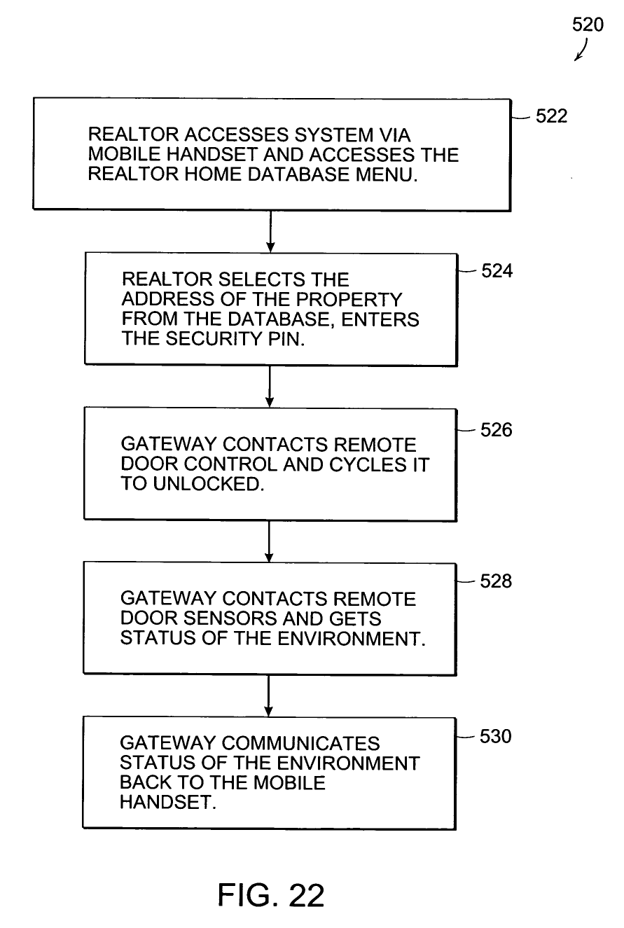

In various embodiments, the GUI's 40, 42, and/or 44 described for FIG. 21 (and FIGS. 22-28) can be implemented based on the GUI's 200, 202, 204, 206, icons 203, 210, 214, 216 and screens 208, 246 described for FIGS. 10, 11, 12, 13, 14, and 15. For example, the multi-environment GUI 40 is implemented as multi-environment GUI 200, the single environment GUI 42 implemented as environment menu 204, and application GUI 44 as application submenu 206.

FIG. 22 is a flowchart that depicts a procedure 520 for real estate door control in a real estate environment 24, according to the principles of the invention. In this procedure 520, a real-estate agent (e.g., the user 62) wants to unlock a door to show a house remotely. The real-estate agent accesses the system 20 via mobile handset (e.g., system enabled mobile communications device 22) and accesses the multi-environment GUI 40 to access the desired environment status menu (e.g., environment GUI 42 or application GUI 44); for example, a real estate database menu (step 522). The real-estate agent selects the address of the property from the database, and enters the security PIN (step 524). The real-estate menu for the real estate environment 24 is configured in various approaches. In one embodiment, the real-estate menu corresponds to a multi-environment GUI 40 that shows icons for different properties or groups of properties (e.g., organized by area) Alternatively, the real-estate menu provides a list of properties that can be scanned, or provides a search mechanism so that the real-estate agent can search for a particular property.

The appropriate gateway 46 in the real-estate environment 24 (e.g., the selected house to be shown) contacts the remote device 52 (e.g., remote door control) and cycles the door to unlock it (step 526). The gateway 46 contacts the remote door sensors and gets status of the remote device 52 (e.g., door locks) in the real-estate environment 24 (step 528). The gateway 46 communicates status of the remote device 52 (e.g., door lock) in the real-estate environment 24 back to the control software 32 for presentation on the display 30 of the mobile communications device 22 (e.g., mobile handset) (step 530).

A process similar to this procedure 520 can be used to lock the door after the house has been shown by a real-estate agent. For example, the real-estate agent (e.g., user 62) commands the gateway 46 to lock the door in step 526, and steps 528 and 530 are adjusted accordingly, so that the control software 32 presents a locked status in the display 30 at the mobile communications device 22 (e.g., mobile handset).

In other embodiments, variations of the procedure 520 are used to accomplish other tasks appropriate for the real-estate environment 24 (e.g., house to be shown), such as checking and adjusting temperature, checking and adjusting lights, turning on or off a lawn irrigation system, and other suitable tasks. The real estate environment 24 can be combined with other environments 24 (e.g., other types of real estate) in a multi-environment GUI 40.

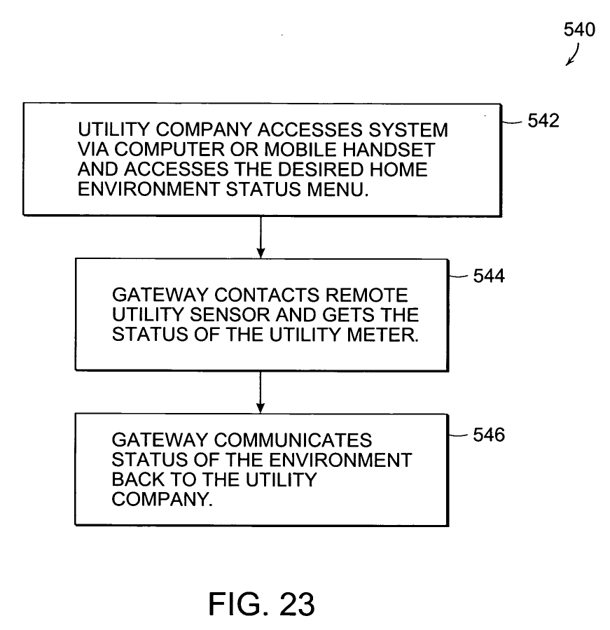

FIG. 23 is a flowchart that depicts a procedure 540 for a meter control and monitoring in a measurement environment 24 (e.g., home environment 24 or utility meter environment 24), according to the principles of the invention. In this procedure 540, a utility company agent (e.g., the user 62) wants to read and control a services meter (e.g., water meter, gas meter, electrical meter, or other utility meter). The utility agent accesses the system 20 via a communications device 22 (and/or computer) and accesses the multi-environment GUI 40 to display the desired environment status menu (e.g., environment menu 42 or application submenu 44) (step 542). In one embodiment, a multi-environment GUI 40 also includes other environment menus 42, such as for home, business facilities, and other suitable environments 24, or is a unified utilities GUI 40 that includes meter environment GUI's 42 for different types of utilities (e.g., gas meter environment menu 42, electric meter environment menu 42, water meter environment menu 42, and other suitable utility environment menus 42).

In one embodiment, the utility agent selects the address of the property from a utility or other database listing sites having meters. The utility database menu for a utility environment 24 is configured in various approaches. In one embodiment, the utility database menu corresponds to an environment menu 42 that shows icons for different properties or groups of properties (e.g., organized by area). Alternatively, the utility menu provides a list of properties that can be scanned, or provides a search mechanism so that the utility agent can search for a particular property.

The gateway 46 contacts the remote application 50 (e.g., including remote utility sensor) and gets status of the remote device 52 (e.g., utility meter) (step 544). The gateway 46 communicates status of the remote device 52 (e.g., utility meter) in the home or utility environment 24 back to the control software 32 for presentation on the display 30 of the communication device 22 (step 546). In the situation described in FIG. 23, the user 62 uses a mobile communications device 22 or a computer (e.g., with appropriate communications capabilities to provide communications over appropriate connections 26).

A process similar to this procedure 540 can be used to adjust the settings of the remote device 52 (e.g., utility meter) or switch the remote device 52 (e.g., utility meter) off in case of emergency of nonpayment. For example, the utility agent (e.g., user 62) commands the gateway 46 to adjust settings for the utility meter in step 544, and step 546 is modified accordingly, so that the control software 32 presents adjusted settings in the display 30 at the communications device 22. In another example, the utility agent (e.g., user 62) commands the gateway 46 to switch off the utility meter in step 544, and step 546 is modified accordingly, so that the control software 32 presents an off status in the display 30 at the communications device 22.

In other approaches, a procedure similar to procedure 540 can be used to perform other tasks, such as controlling and monitoring various types of metering and measurement devices, such as scientific measurement devices, weather measurement devices, vehicular traffic measurement devices, and other suitable meters and measurement devices.

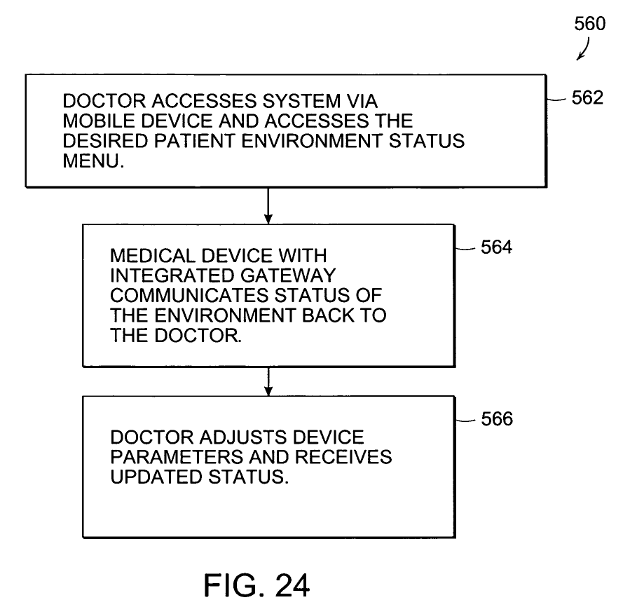

FIG. 24 is a flowchart that depicts a procedure 560 for controlling and monitoring a medical device in a patient environment 24, according to the principles of the invention. In this procedure 560, a doctor (e.g., the user 62) would like to monitor a patient's status remotely and adjust medical device parameters, if needed. The doctor accesses the system 20 via mobile handset (e.g., system enabled mobile communications device 22) and accesses the multi-environment GUI 40 to display the desired patient environment status menu (e.g., environment menu 42 or application submenu 44); for example, showing medical device status for one or more medical devices) (step 562). For example, the doctor accesses the patient environment menu 42 from a multi-environment menu 40 that provides access to a number of patients. In one embodiment, a multi-environment GUI 40 also includes other environment menus 42, such as an environment 24 for each of several medical facilities, which each have some of the doctor's patients. In one embodiment, the patient status environment menu corresponds to a multi-environment menu 40 or an environment menu 42 that shows icons for different patients, groups of patients (e.g., organized by diagnosis), or other organizational approach. Alternatively, a patient environment menu provides a list of patients that can be scrolled, or provides a search mechanism so that the doctor can search for a particular patient.

The remote device 52 (e.g., medical device) with integrated gateway 46 communicates status of the environment 24 back to the control software 32 for presentation to the doctor on the display 30 of the mobile communications device 22 (step 564). The doctor adjusts device parameters and receives updated status (step 566). For example, the doctor adjusts the device parameters by entering and/or selecting commands via the GUI 40, 42, and/or 44 presented by the control software 32 on the display 30. The gateway 46 associated with the remote device 52 (e.g., medical device) communicates the parameter adjustments, and communicates back to the control software 32 to present the status (e.g., changed parameters) in the display 30 at the mobile communications device 22 (e.g., mobile handset).

The doctor can use a similar procedure as procedure 560 to signal for help in case of emergency, by receiving the status of the remote device 52 (e.g., medical device), and then accessing a help communication screen (e.g., by selecting a communication or emergency icon or option) which can be accessed via the GUI's 40, 42, and/or 44 to initiate communication to a source of help (e.g., initiate a cellular telephone call to the appropriate nurses' station at a hospital).

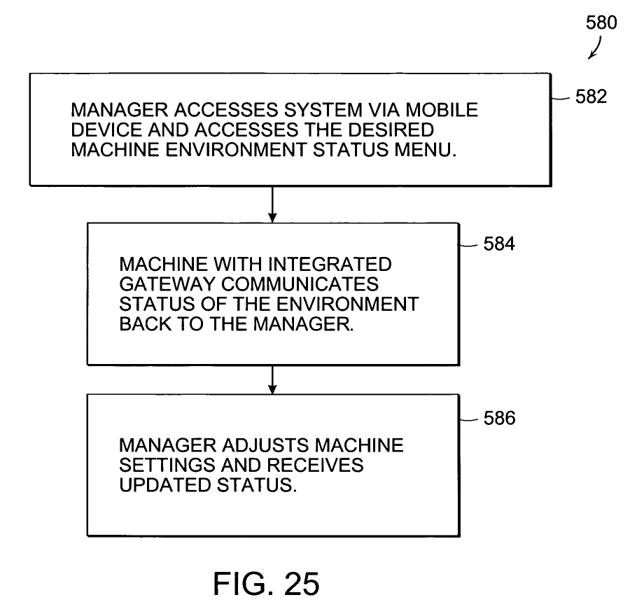

FIG. 25 is a flowchart that depicts a procedure 580 for controlling and monitoring a mechanical device 52 in a machine environment 24, according to the principles of the invention. In this procedure 580, a manager (e.g., the user 62) would like to monitor the status of a remote device 52 (e.g., mechanical device or machine) and adjust its settings, if needed. The manager accesses the system 20 via a communications device 22 or a computer and accesses the multi-environment GUI 40 to display the desired machine environment status menu (e.g., using the environment menu 42 or application submenu 44) (step 582). For example, the manager accesses the machine environment menu 42 from a multi-environment menu 44 that provides access to a number of machines (e.g., as occurs in a factory). In one embodiment, a multi-environment GUI 40 also includes other environment menus 42, such as for an environment 24 for each of several factories, which each have machines 52. In one embodiment, the machine environment status menu corresponds to a multi-environment menu 40 or an environment menu 42, or application menu 44 that shows icons for different machines, groups of machines (e.g., organized by related functions), or other organizational approach. Alternatively, a machine environment menu 42 provides a list of machines that can be scanned, or provides a search mechanism so that the manager can search for a particular machine.

The remote device 52 (e.g., machine) with integrated gateway 46 communicates status of the environment 24 back to the control software 32 for presentation to the manager on the display 30 of the communications device 22 or computer (step 584). The manager adjusts machine settings and receives updated status (step 586). For example, the manager adjusts the device settings by entering and/or selecting commands via the GUI 40, 42, and/or 44 presented by the control software 32 on the display 30. The gateway 46 associated with the remote device 52 (e.g., machine) communicates the settings, and communicates back to the control software 32 to present the status (e.g., changed settings) in the display 30 at the mobile communications device 22. Various kinds of multi-environment GUI's 40 can be implemented; for example, a factory multi-environment GUI 40 that provides access to a HVAC environment 42 GUI, a security environment GUI 42, and one or more machine environment GUI's 42.

The manager can use a similar procedure as procedure 580 to signal for help in case of emergency, by receiving the status of the remote device 52 (e.g., machine), and then displaying a help communication screen (e.g., by selecting a communication or emergency icon or option) accessed via the GUI's 40, 42, and/or 44 to initiate communication to a source of help (e.g., initiate a cellular telephone call to the appropriate factory supervisor or control room for a factory).

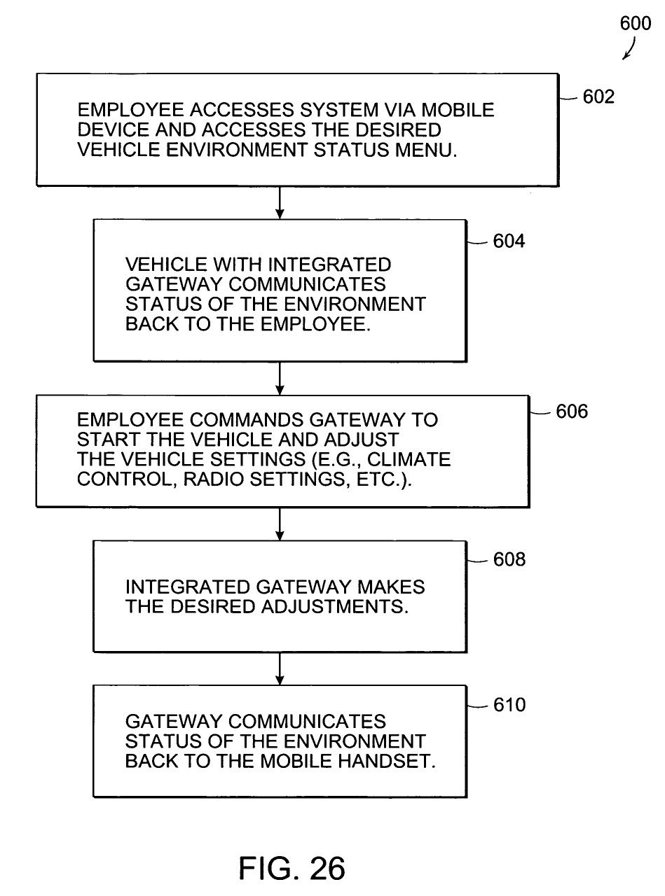

FIG. 26 is a flowchart that depicts a procedure 600 for vehicle control in a rental environment 24, according to the principles of the invention. In this procedure 600, a car rental employee (e.g., the user 62) wishes to adjust vehicle settings for the comfort and convenience of a customer. The car rental employee accesses the system 20 via a communications device 22 or computer, and accesses the multi-environment GUI 40 to display the desired environment status menu (e.g., environment menu 42 or application submenu 44); for example, the employee accesses a vehicle environment menu 42 from a multi-environment menu 40. The owner then accesses the desired vehicle environment status menu (for example, showing door lock status and other vehicle device features) (step 602). In one embodiment, the vehicle environment status menu is the same as the vehicle environment menu 42. In another embodiment, the vehicle environment status menu is a multiple icon application submenu 44. In one embodiment, a multi-environment GUI 40 also includes other environment menus 42, such as for other kinds of rental equipment (e.g., trucks, trailers, and other suitable equipment). In one embodiment, the multi-environment menu 40 that shows icons for different vehicles or groups of vehicles (e.g., organized by type) Alternatively, the multi-environment menu 40 provides a list of vehicles that can be scanned, or provides a search mechanism so that the employee can search for a particular vehicle.

The integrated gateway 46 associated with a vehicle sends the status of the environment 24 back to the control software 32 for presentation on the display 30 of the communications device 22 (step 604). The rental car employee (e.g., user 62) commands the gateway 46 to perform tasks, such as starting the vehicle and adjusting the vehicle settings (e.g., climate control, radio settings, and other suitable settings) by entering the command via the GUI 40, 42, and/or 44 presented by the control software 32 on the display 30 (step 606). The integrated gateway 46 associated with the vehicle contacts one or more remote applications 50 in the rental environment 24 to make the desired adjustments in the vehicle settings (step 608). The gateway 46 communicates the status of the environment 24 back to the control software 32 to present the status (e.g., adjusted settings) in the display 30 at the communications device 22 (step 610). A process similar to this procedure 600 can be used to monitor the vehicle status or track the asset.

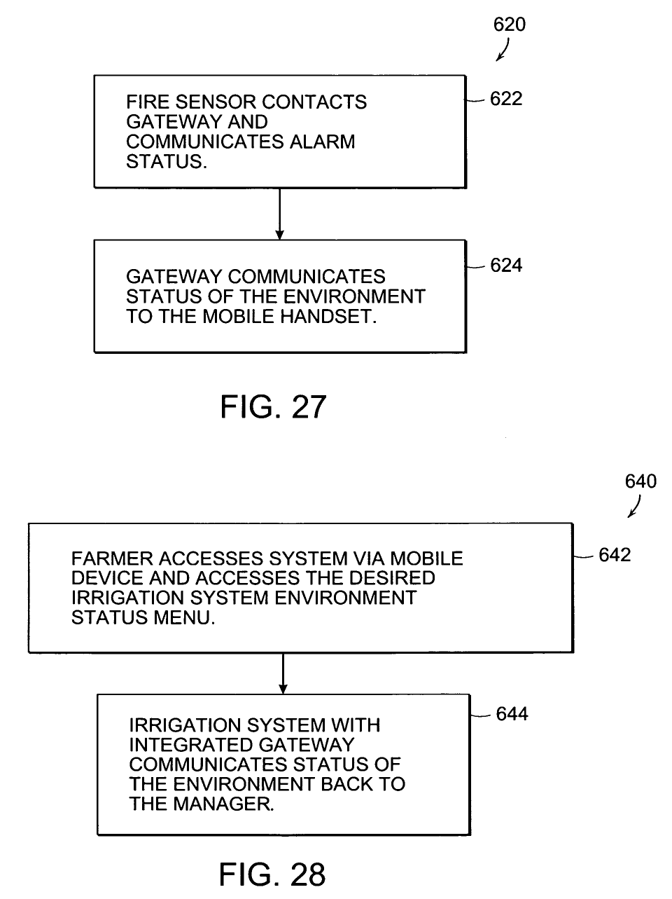

FIG. 27 is a flowchart that depicts a procedure 620 for a fire alarm notification in a building environment 24, according to the principles of the invention. In this procedure 620, a building owner (e.g., the user 62) wants to be notified in the event of a fire. In one embodiment, a multi-environment GUI 40 also includes other environment menus 42, such as for the owner's home, business, and other suitable environments, which are capable of displaying a fire alarm status for the various environments 24. The fire alarm status (e.g., icon and/or alarm screen) is displayed immediately to the owner (that is, without requiring the owner to access various menus and icons in the GUI's 40, 42, 44).

The fire sensor at a building contacts the gateway 46 for that building and communicates an alarm status (step 622). The gateway 46 communicates the status of the environment 24 (e.g., fire alarm) back to the control software 32 for presentation on the display 30 of the communications device 22 (e.g., mobile handset) (step 624). A process similar to this procedure 620 can also initiate an automatic emergency call to authorities from the building gateway 46. In other embodiments, variations of the procedure 620 are used to accomplish other alarm tasks, such as an alarm for a vehicle break-in or fire.

FIG. 28 is a flowchart that depicts a procedure 640 for controlling and monitoring equipment in a farm environment 24, according to the principles of the invention. In this procedure 640, a farmer (e.g., the user 62) would like to monitor the status of a remote device 52 (e.g., irrigation system) and adjust its settings, if needed. The farmer accesses the system 20 via a mobile communications device 22 or computer, and accesses the multi-environment GUI 40 to display the desired farm environment status menu (e.g., environment menu 42 or application submenu 44) (step 642). For example, the farmer accesses the irrigation environment menu 42 from a multi-environment menu 40 that provides access to one or more remote devices 52 that control the irrigation equipment. In one embodiment, a multi-environment GUI 40 also includes other environment menus 42, such as for an environment 24 for each of several different types of farm equipment and/or farm building monitoring and control environments. In another embodiment, a multi-environment GUI 40 includes environment GUI's 42 for various water management related services and facilities, such as an irrigation environment 24, a water meters utility environment 24, and a water flow (e.g., dam) control environment 24.

The remote device 52 (e.g., irrigation equipment) with integrated gateway 46 communicates status of the environment 24 back to the control software 32 for presentation to the farmer on the display 30 of the communications device 22 (step 644).

The farmer can use a similar procedure as procedure 640 to adjust the settings of the irrigation system, turning it on, or off, depending on conditions, and/or adjust settings for other farm equipment. For example, the farmer adjusts the device settings by entering and/or selecting commands via the GUI's 40, 42, and/or 44 presented by the control software 32 on the display 30. The gateway 46 associated with the remote device 52 (e.g., irrigation and/or other farm equipment) communicates the settings, and communicates back to the control software 32 to present the status (e.g., changed settings) in the display 30 at the communications device 22.

In various embodiments, various environments 24 can be combined into multi-environment GUI's 40 in any suitable combinations. In various embodiments, a multi-environment menu 40 can be based on combinations of any two or more environments 24 described herein. For example, an automotive environment menu 42 can be combined with a medical environment menu 42 to form a multi-environment menu 40 for an ambulance.

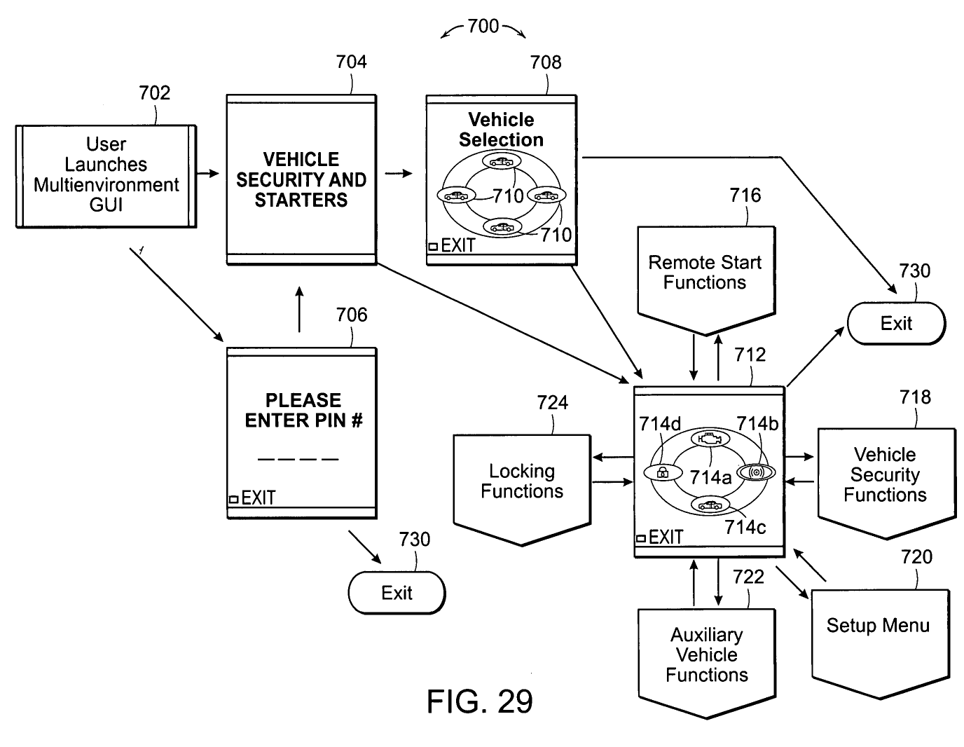

FIG. 29 is an illustration that depicts a multi-environment vehicle graphic user interface (GUI) 700, according to the principles of the invention. The multi-environment vehicle GUI 700 (an embodiment of a multi-environment GUI 40) includes user launch 702, opening screen 704, security screen 706, multi-environment vehicle screen 708, vehicle environment screen 712, and exit option 730. The vehicle environment screen 708 (an embodiment of an environment GUI 42) includes vehicle icons 710 associated with vehicle environments 24 for four vehicles. The vehicle environment screen 712 includes icons for applications 50, including remote start icon 714a, security icon 714b, auxiliary functions icon 714c, and locking functions icon 714d (generally referred to as “functions icons” 714). The vehicle environment screen 712 provides access to remote start functions 716 (using the remote start icon 714a), vehicle security functions 718 (using the security icon 714b), setup menu 720 (accessed by a “softkey” on the mobile communications device 22 associated with text such as “Menu” on the lower part of the display), auxiliary vehicle functions 722 (using the auxiliary functions icon 714c) and locking functions 724 (using the locking functions icon 714d). In one embodiment, the functions submenus 716, 718, 722, and 724 are implementations of the application GUI 44. The setup menu 720 provides access to administrative and setup functions such as changing the PIN or accessing help information for the user 62.

In a summary, the user 62 launches the multi-environment GUI 702 (e.g., from an icon or menu provided for the display 30 of the mobile communications device 22). In one embodiment, the user 62 is required to complete the security screen 706 (e.g., by entering a PIN). The control software 32 presents the MCMS opening screen 704. The user 62 then accesses the multi-environment vehicle screen 708, and selects one of the vehicle icons 710 to present the vehicle environment screen 712 for the selected vehicle. The user 62 then has the option of selecting one of the functions icons 714 to access the relevant functions submenus 716, 718, 722, and 724.

In one embodiment, a computer program product including a computer readable medium (e.g., one or more of DVD's, CD's, diskettes, tapes, and/or other suitable medium) provides software instructions for one or more of the software components (e.g., that implement the multi-environment GUI 40, single environment GUI's 42, and application GUI's 44) of the control software 32. The computer program product can be installed by downloading from a web site (e.g., 64) associated with a server (e.g., administrative server 66 and/or communications server 68) or by any suitable software installation procedure. In another embodiment, a computer program propagated signal product embodied on a propagated signal on a propagation medium (e.g., a radio wave, an optical wave, other electromagnetic wave, a sound wave, or an electrical signal propagated over the communications connections 26, Internet, and/or other network) provides software instructions for one or more of the components (e.g., that implement one or more of the GUI's 40, 42, 44) of the control software 32. Alternatively, the propagated signal is an analog carrier wave or a digital signal carried on the propagated medium. The propagated signal can be a digitized signal propagated over the communications connections 26, or other wireless communications or network. The propagated signal is a signal that can be transferred over the propagation medium over a period of time, such as the instructions for a software component, program, object, procedure, or other software entity sent in segments (e.g., packets) over the communications connections 26 or wireless network over a period of seconds, minutes, or longer.

While the invention has been shown and described with reference to specific preferred embodiments, it should be understood by those skilled in the art that various changes in form and detail may be made therein without departing from the spirit and scope of the invention as defined by the following claims.

For example, the processors 34, 57, 72, 82 and circuitry of the mobile communications device 22, gateway 46, the administrative server 66, the communications server 68 and other devices (e.g., functional modules 48) can be based on analog, nanotechnology, biological, quantum, optical, or other suitable computing technologies, or hybrid technologies including two or more of the above technologies, as well as computing technologies to be developed in the future.

For example, all or part of the software functions (e.g., mobile device control software 32, gateway software 58, administrative server software 74, and/or communication server software 84) are implemented in hardware, for example, in a programmable gate array (PGA), programmable logic device (PLD), application specific integrated circuit (ASIC), or other suitable IC chip. Portions of the software functions (e.g.,32, 58, 74, and/or 84) can be implemented by plural IC chips communicating over at least one bus and/or by a multifunction “system on a chip” IC.

In another example, the functions of the invention (e.g., as implemented for the GUI's 40, 42, 44 of the control software 32) as described herein can be performed by two or more processors (e.g., 34, 57, 72, 82, and/or other suitable processors) distributed in different locations using a distributing computing (e.g., distributed object) approach through a network, the Internet, communications connections 26, or other suitable connections. For example, one processor (e.g., 34) of one digital device (e.g., 22) can execute the instructions of a component that implements the multi-environment GUI 40 and one or more other processors (e.g., 57, 72, and/or 82) of an electronic device (e.g., 46, 66, and/or 68) can execute the instructions of other components (e.g. that implement GUI's 42, 44) of the control software 32. Generally, different combinations of components (e.g., that implement the GUI's 40, 42, and/or 44) can operate on different processors (34, 57, 72, 82, and/or other suitable processors) located on the same or different electronic devices (22, 46, 66, 68, and/or other suitable devices) connected by communications connections 26 and/or networks.

What is claimed is:

1. A method for controlling, monitoring, and using remote applications over communications connections to a mobile communications device, the method comprising:
providing a multi-environment menu to a display of the mobile communications device, the multi-environment menu presenting one or more environment icons, each environment comprising one or more remote applications providing access to one or more remote devices;
providing a selected environment menu to the display of the mobile communications device based on a selection of one of the presented environment icons, the selected environment menu presenting one or more application icons;
providing a selected application submenu to the display of the mobile communications device based on a selection of one of the presented application icons, the selected application submenu presenting one or more remote device icons; and
initiating one or more messages from the mobile communications device to a selected remote device to invoke one or more remote actions based on a selection of one of the presented remote device icons.

2. The method of claim 1, further comprising providing a selected remote device submenu to the display of the mobile communications device based on the selected remote device icon, the selected remote device submenu presenting icons for the one or more remote actions, and initiating one or more messages from the mobile communications device comprises initiating one or more messages to the selected remote device based on a selection of one of the presented remote action icons.

3. The method of claim 1, further comprising providing a response on the display of the mobile communications device received from the selected remote device in response to the one or more messages initiated from the mobile communications device to the selected remote device.

4. The method of claim 1, further comprising providing an alert to the display of the mobile communications device in response to one of the remote devices initiating the alert.

5. The method of claim 1, wherein initiating one or more messages from the mobile communications device comprises transferring the one or more messages to a gateway associated with a selected remote application indicated by the selected application icon.

6. The method of claim 1, wherein initiating one or more messages from the mobile communications device comprises transferring the one or more messages to a server for transfer to a selected remote application indicated by the selected application icon.

7. The method of claim 1, wherein the one or more environment icons comprise one or more of an automotive environment icon, a home environment icon, a business environment icon, a real estate environment icon, a remote water meter environment icon, a remote gas meter environment icon, a medical device environment icon, a machinery environment icon, a rental vehicle environment icon, a fire alarm environment icon, and a farm irrigation environment icon.

8. The method of claim 1, wherein the one or more application icons comprise one or more of a door lock application icon, an ignition switch application icon, an electrical outlet application icon, an environmental control application icon, a climate control application icon, a security control application icon, a video camera application icon, a water meter application icon, a gas meter application icon, a medical device application icon, a mechanical device application icon, a rental vehicle application icon, a fire alarm application icon, and a farm irrigation application icon.

9. The method of claim 1, wherein the mobile communications device comprises one or more navigation controls and one or more selection controls, and further comprising:
operating one of the navigation controls to navigate to one of the presented environment icons, and operating one of the selection controls to select that presented environment icon;
operating one of the navigation controls to navigate to one of the presented application icons, and operating one of the selection controls to select that presented application icon; and
operating one of the navigation controls to navigate to one of the presented remote device icons, and operating one of the selection controls to select that presented remote device icon.

10. A server accessing a mobile communications device over communications connections, the server comprising:
control software for controlling, monitoring, and using remote applications, the control software comprising instructions;
a processor for executing the instructions of the control software; and
a communications module:
wherein the processor is configured by the instructions of the control software to provide a multi-environment menu to a display of the mobile communications device, the multi-environment menu presenting one or more environment icons, each environment comprising one or more remote applications providing access to one or more remote devices; to provide a selected environment menu to the display of the mobile communications device based on a selection of one of the presented environment icons, the selected environment menu presenting one or more application icons; and to provide a selected application submenu to the display of the mobile communications device based on a selection of one of the presented application icons, the selected application submenu presenting one or more remote device icons for invoking one or more remote actions based on a selection of one of the presented remote device icons; and
the communications module receives one or more messages initiated from the mobile communications device and transfers one of the multi-environment menu, the environment menus, and application submenus to the display of the mobile communications device in response to the one or more received messages.

11. The server of claim 10, wherein the server is one of an administrative server and a communications server.

12. A server accessing a mobile communications device over communications connections;
the server comprising:
means for controlling and monitoring remote applications; and
a communications module:
wherein the means for controlling and monitoring remote applications provides a multi-environment menu to a display of the mobile communications device, the multi-environment menu presenting one or more environment icons, each environment comprising one or more remote applications providing access to one or more remote devices; provides a selected environment menu to the display of the mobile communications device based on a selection of one of the presented environment icons, the selected environment menu presenting one or more application icons; and provides a selected application submenu to the display of the mobile communications device based on a selection of one of the presented application icons, the selected application submenu presenting one or more remote device icons for invoking one or more remote actions based on a selection of one of the presented remote device icons; and
the communications module receives one or more messages initiated from the mobile communications device and transfers one of the multi-environment menu, the environment menus, and application submenus to the display of the mobile communications device in response to the one or more received messages.

13. The server of claim 12, wherein the means for controlling and monitoring remote applications provides a selected remote device submenu to the display of the mobile communications device based on the selected remote device icon, the selected remote device submenu presenting icons for the one or more remote actions; and the communications module receives the one or more messages to the selected remote device based on a selection of one of the presented remote action icons.

14. The server of claim 12, wherein the means for controlling and monitoring remote applications provides a response on the display of the mobile communications device received from the selected remote device in response to the one or more messages initiated from the mobile communications device to the selected remote device.

15. The server of claim 12, wherein the means for controlling and monitoring remote applications provides an alert to the display of the mobile communications device in response to one of the remote devices initiating the alert.

16. The server of claim 12, wherein the communications module transfers the one or more messages to a gateway associated with a selected remote application indicated by the selected application icon.

17. The server of claim 12, wherein the one or more environment icons comprise one or more of an automotive environment icon, a home environment icon, a business environment icon, a real estate environment icon, a remote water meter environment icon, a remote gas meter environment icon, a medical device environment icon, a machinery environment icon, a rental vehicle environment icon, a fire alarm environment icon, and a farm irrigation environment icon.

18. The server of claim 12, wherein the one or more application icons comprise one or more of a door lock application icon, an ignition switch application icon, an electrical outlet application icon, an environmental control application icon, a climate control application icon, a security control application icon, a video camera application icon, a water meter application icon, a gas meter application icon, a medical device application icon, a mechanical device application icon, a rental vehicle application icon, a fire alarm application icon, and a farm irrigation application icon.

19. A computer program propagated signal product embodied in a propagated medium having instructions for control software for controlling, monitoring, and using remote applications, such that the instructions, when carried out by a processor of a mobile communications device, configure the mobile communications device to perform the steps of:
providing a multi-environment menu to a display of the mobile communications device, the multi-environment menu presenting one or more environment icons, each environment comprising one or more remote applications providing access to one or more remote devices;
providing a selected environment menu to the display of the mobile communications device based on a selection of one of the presented environment icons, the selected environment menu presenting one or more application icons;
providing a selected application submenu to the display of the mobile communications device based on a selection of one of the presented application icons, the selected application submenu presenting one or more remote device icons; and
initiating one or more messages from the mobile communications device to a selected remote device to invoke one or more remote actions based on a selection of one of the presented remote device icons.

20. The computer program propagated signal product of claim 19, wherein the instructions configure the mobile communications device to perform the steps of providing a selected remote device submenu to the display of the mobile communications device based on the selected remote device icon, the selected remote device submenu presenting icons for the one or more remote actions, and initiating one or more messages from the mobile communications device comprises initiating one or more messages to the selected remote device based on a selection of one of the presented remote action icons.

21. The computer program propagated signal product of claim 19, wherein the instructions configure the mobile communications device to perform the step of providing a response on the display of the mobile communications device received from the selected remote device in response to the one or more messages initiated from the mobile communications device to the selected remote device.

22. The computer program propagated signal product of claim 19, wherein the instructions configure the mobile communications device to perform the step of providing an alert to the display of the mobile communications device in response to one of the remote devices initiating the alert.

23. The computer program propagated signal product of claim 19, wherein the instructions configure the mobile communications device to perform the step of transferring the one or more messages to a gateway associated with a selected remote application indicated by the selected application icon.

24. The computer program propagated signal product of claim 19, wherein the instructions configure the mobile communications device to perform the step of transferring the one or more messages to a server for transfer to a selected remote application indicated by the selected application icon.

25. The computer program propagated signal product of claim 19, wherein the one or more environment icons comprise one or more of an automotive environment icon, a home environment icon, a business environment icon, a real estate environment icon, a remote water meter environment icon, a remote gas meter environment icon, a medical device environment icon, a machinery environment icon, a rental vehicle environment icon, a fire alarm environment icon, and a farm irrigation environment icon.

26. The computer program propagated signal product of claim 19, wherein the one or more application icons comprise one or more of a door lock application icon, an ignition switch application icon, an electrical outlet application icon, an environmental control application icon, a climate control application icon, a security control application icon, a video camera application icon, a water meter application icon, a gas meter application icon, a medical device application icon, a mechanical device application icon, a rental vehicle application icon, a fire alarm application icon, and a farm irrigation application icon.

27. A method for controlling, monitoring, and using remote applications over communications connections to a server, the method comprising:
receiving at a server one or more messages from a mobile communications device based on a selection of a remote device icon from a selected application submenu selected from one or more application icons provided by an environment menu selected from one or more environment icons provided by a multi-environment menu, presented on a display of the mobile communications device;
transferring the one or more received messages to a destination associated with a remote device indicated by the selected remote device icon;
receiving at the server a response based on the one or more received messages transferred to the destination; and
transferring the received response to the mobile communication device.

28. The method of claim 27, further comprising providing the received response to the display of the mobile communications device.

29. The method of claim 27, wherein the server is one of an administrative server and a communications server.

30. The method of claim 27, wherein the destination is one of a mobile communication device, a gateway, an application, and a remote device.

31. The method of claim 27, further comprising receiving at the server an alert initiated by the destination and transferring the alert to the mobile communications device.

32. The method of claim 27, wherein transferring the one or more received messages comprises transferring the one or more received messages to a gateway associated with the selected application menu indicated by the selected application icon, the gateway providing access to one or more remote applications having one or more remote devices.

33. The method of claim 27, wherein the remote device icon is one of a door lock icon, an ignition switch icon, an electrical icon, a climate control icon, a video camera icon, a water meter icon, a gas meter icon, a medical device icon, a mechanical device icon, a rental vehicle icon, a fire alarm icon, and a farm irrigation icon.

34. The method of claim 27, wherein the one or more environment icons comprise one or more of an automotive environment icon, a home environment icon, a business environment icon, a real estate environment icon, a remote water meter environment icon, a remote gas meter environment icon, a medical device environment icon, a machinery environment icon, a rental vehicle environment icon, a fire alarm environment icon, and a farm irrigation environment icon.

35. The method of claim 27, wherein the one or more application icons comprise one or more of a door lock application icon, an ignition switch application icon, an electrical outlet application icon, an environmental control application icon, a climate control application icon, a security control application icon, a video camera application icon, a water meter application icon, a gas meter application icon, a medical device application icon, a mechanical device application icon, a rental vehicle application icon, a fire alarm application icon, and a farm irrigation application icon.

36. A server accessing a mobile communications device over communications connections, the server comprising a processor and instructions for managing messages for controlling, monitoring, and using remote applications, the instructions for managing messages configuring the processor to:
receive one or more messages from a mobile communications device based on a selection of a remote device icon from a selected application submenu selected from one or more application icons provided by an environment menu selected from one or more environment icons provided by a multi-environment menu, presented on a display of the mobile communications device;
transfer the one or more received messages to a destination associated with a remote device indicated by the selected remote device icon;
receive at the server a response based on the one or more received messages transferred to the destination; and
transfer the received response to the mobile communication device.

37. The server of claim 36, wherein the instructions for managing messages configure the processor to provide the received response to the display of the mobile communications device.

38. The server of claim 36, wherein the server is one of an administrative server and a communications server.

39. The server of claim 36, wherein the destination is one of a mobile communication device, a gateway, an application, and a remote device.

40. The server of claim 36, wherein the instructions for managing messages configure the processor to receive at the server an alert initiated by the destination and to transfer the alert to the mobile communications device.

41. The server of claim 36, wherein the remote device icon is one of a door lock icon, an ignition switch icon, an electrical icon, a climate control icon, a video camera icon, a water meter icon, a gas meter icon, a medical device icon, a mechanical device icon, a rental vehicle icon, a fire alarm icon, and a farm irrigation icon.

42. The server of claim 36, wherein the one or more environment icons comprise one or more of an automotive environment icon, a home environment icon, a business environment icon, a real estate environment icon, a remote water meter environment icon, a remote gas meter environment icon, a medical device environment icon, a machinery environment icon, a rental vehicle environment icon, a fire alarm environment icon, and a farm irrigation environment icon.

43. The server of claim 36, wherein the one or more application icons comprise one or more of a door lock application icon, an ignition switch application icon, an electrical outlet application icon, an environmental control application icon, a climate control application icon, a security control application icon, a video camera application icon, a water meter application icon, a gas meter application icon, a medical device application icon, a mechanical device application icon, a rental vehicle application icon, a fire alarm application icon, and a farm irrigation application icon.
